(19) United States
(12) Patent Application Publication
(10) Pub. No.: US 2022/0193410 A1
(43) Pub. Date: Jun. 23, 2022

(54) METHODS FOR TREATING OR REDUCING OCULAR PAIN

(71) Applicant: United States Government As Represented By The Department of Veterans Affairs, Washington, DC (US)

(72) Inventor: Anat Galor, Miami, FL (US)

(21) Appl. No.: 17/602,058

(22) PCT Filed: Apr. 8, 2020
(86) PCT No.: PCT/US2020/027190

§ 371 Date: Oct. 7, 2021

Related U.S. Application Data
(60) Provisional application No. 62/830,900, filed on Apr. 8, 2019.

(51) Int. Cl. A61N 1/36; A61K 38/13; A61K 38/20

(52) U.S. Cl. CPC A61N 1/36021; A61K 38/2006; A61K 38/13

(57) ABSTRACT

The disclosure relates to methods of treating or reducing ocular pain in a subject. The method includes stimulating nasal tissue in a subject in need of treatment. Optionally, the method can be performed using a stimulator probe having first and second nasal insertion prongs having respective electrodes that can be positioned adjacent to a septum of the subject. The stimulator probe can be connected to a stimulator body having a control subsystem to control an electrical stimulus to be delivered to the subject via the stimulator probe. Optionally, the subject does not have low tear volume.

### CROSS-REFERENCE TO RELATED APPLICATION

This application claims priority to and the benefit of the filing date of U.S. Provisional Patent Application No. 62/830,900, filed Apr. 8, 2019, which is incorporated herein by reference in its entirety.

### STATEMENT REGARDING FEDERALLY SPONSORED RESEARCH

This invention was made with government support under grant no. P30EY014801 awarded by the National Institutes of Health. The government has certain rights in the invention.

### BACKGROUND

Many treatments are available for treating ocular pain, but none provides substantial efficacy for treatment. Thus, useful methods for treating and reducing ocular pain are needed.

### SUMMARY

Disclosed herein are methods for reducing ocular pain in a subject, the methods comprising electrically stimulating nasal tissue of the subject.

Disclosed herein are methods for treating ocular pain in a subject, the methods comprising electrically stimulating nasal tissue of the subject.

Disclosed herein are methods of reducing ocular burning and/or ocular stinging in a subject, the methods comprising electrically stimulating nasal tissue of the subject.

Disclosed herein are methods of reducing or ameliorating one or more signs, symptoms, causes or effects of ocular pain in a subject, the methods comprising electrically stimulating nasal tissue of the subject.

Other features and advantages of the present compositions and methods are illustrated in the description below, the drawings, and the claims.

### DESCRIPTION OF THE DRAWINGS

These and other features of the preferred embodiments of the invention will become more apparent in the detailed description in which reference is made to the appended drawings wherein:

FIG. 1 is a schematic of a system for treating ocular pain in accordance with embodiments disclosed herein.

FIG. 2 is a schematic of an underside of a nose of a subject and a stimulator probe of the system of FIG. 1.

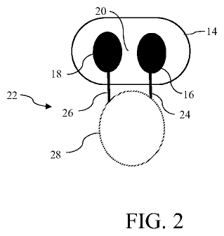

### DETAILED DESCRIPTION

The present disclosure can be understood more readily by reference to the following detailed description of the invention, the figures and the examples included herein.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein (including those within the provided reference lists) are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

Further, the dates of publication provided herein can be different from the actual publication dates, which can require independent confirmation.

As used in the specification and the appended claims, the singular forms “a,” “an” and “the” include plural referents unless the context clearly dictates otherwise.

The word “or” as used herein means any one member of a particular list and also includes any combination of members of that list.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

Optionally, in some aspects, when values are approximated by use of the antecedents “about,” “substantially,” “approximately,” or “generally,” it is contemplated that values within up to 15%, up to 10%, up to 5%, or up to 1% (above or below) of the particularly stated value or characteristic can be included within the scope of those aspects.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, the subject is a human. The term does not denote a particular age or sex. Thus, adult, child, adolescent and newborn subjects, as well as fetuses, whether male or female, are intended to be covered.

As used herein, the term “patient” refers to a subject afflicted with ocular pain or a disease, disorder or condition associated with ocular pain. The term “patient” includes human and veterinary subjects. In some aspects of the disclosed methods, the “patient” has been diagnosed with a need for treatment for ocular pain, such as, for example, prior to the administering step.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be ocular pain.

As used herein, “treating” can also refer to the medical management of a patient with the intent to cure, ameliorate, stabilize, or prevent a disease, condition, or disorder. This term includes active treatment, that is, treatment directed specifically toward the improvement of a disease, condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, condition, or disorder; preventative treatment, that is, treatment directed to minimizing or partially or completely inhibiting the development of the associated disease, condition, or disorder; and supportive treatment, that is, treatment employed to supplement another specific therapy directed toward the improvement of the associated disease, condition, or disorder. In various aspects, the term covers any treatment of a subject, including a mammal (e.g., a human), and includes: (i) preventing, for example, ocular pain from occurring in a subject that can be predisposed to ocular pain but has not yet been diagnosed as having it; (ii) inhibiting ocular pain, i.e., arresting its development; or (iii) relieving pain, i.e., causing regression of the ocular pain.

As used herein, the terms “inhibit” or “inhibiting” mean decreasing or reducing ocular pain that would occur without treatment and/or causing one or more symptoms of ocular pain to decrease.

The terms “administering” and “administration” refer to any method of providing a pharmaceutical preparation to a subject. Such methods are well known to those skilled in the art and include, but are not limited to, oral administration, sublingual administration, trans-buccal mucosa administration, transdermal administration, administration by inhalation, nasal administration, topical administration, intravaginal administration, ophthalmic administration, intraaural administration, intracerebral administration, intrathecal administration, rectal administration, intraperitoneal administration, and parenteral administration, including injectable such as intravenous administration, intra-arterial administration, intramuscular administration, intradermal administration, and subcutaneous administration. Ophthalmic administration can include topical administration, subconjunctival administration, sub-Tenon's administration, epibulbar administration, retrobulbar administration, intra-orbital administration, and intraocular administration, which includes intra-vitreal administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “ocular” means the eye, surrounding tissues of the eye, and bodily fluids in the region of the eye. Specifically, the term includes the cornea or, the sclera or, the uvea, the conjunctiva (e.g., bulbar conjunctiva, palpebral conjunctiva, and tarsal conjunctiva), anterior chamber, lacrimal sac, lacrimal canals, lacrimal ducts, medial canthus, nasolacrimal duct, and the eyelids (e.g., upper eyelid and lower eyelid). Additionally, the term includes the inner surface of the eye (conjunctiva overlying the sclera), and the inner surface of the eyelids (e.g., the palpepral conjunctiva).

Dry eye (DE) is a common condition that is associated with significant ocular morbidity.¹ It is a multifactorial disease comprised of tear film and ocular surface disturbances such as aqueous insufficiency, evaporative deficiency and/or somatosensory dysfunction which often overlap and interact.² These mechanisms result in symptoms which include ocular pain, described in terms of “dryness”, “burning”, and “aching” and visual disturbances. Treatments for DE aim to improve tear health by one or more of: increasing tear volume with artificial tears, decreasing inflammation with corticosteroids, cyclosporine, or lifitegrast, addressing meibomian gland dysfunction with lid hygiene and antibiotics, or blocking the outflow of tears with punctal closure.3 Alpha 2 delta (α2δ) ligands (gabapentin and pregabalin)⁴, autologous serum tears⁵, and botulinum toxin injection^{6, 7} have been used in individuals with DE symptoms in the setting of presumed somatosensory dysfunction (i.e. neuropathic mechanisms). However, results can be variable and refractory in some cases.

Recently, a new device was approved for the treatment of low tear volume which targets the neurophysiology of the lacrimal functional unit. TrueTear® (Allergan, San Diego, Calif.) stimulates the anterior ethmoidal nerve with adjustable tiny pulses of energy, up to a maximum of 13 V or 5 mA in 30-60 Hz, which kindles the naso-lacrimal reflex.⁸ The anterior ethmoidal nerve in the nasal cavity is an extraconal branch of the nasociliary nerve, a branch of ophthalmic division of the trigeminal nerve which initiates the afferent limb of the nasolacrimal reflex.⁹ The efferent limb of the naso-lacrimal reflex originates from the superior salivary nucleus along the facial nerve (intermedius), through the geniculate ganglion, the greater superficial petrosal nerve, the nerve of the pterygoid canal to the sphenopalatine ganglion (SPG) and via the zygomatic nerve to the lacrimal nerve and the lacrimal gland.¹⁰ While reflex aqueous tear secretion is predominantly regulated by this parasympathetic arc, sympathetic nerves from the superior cervical ganglion (SCG) also affect aqueous production and the composition of tear proteins and electrolytes.¹¹⁻¹³ While the innervation pattern of the meibomian glands (for lipids) and globet cells (for mucin) are less well characterized¹⁰, animal¹⁴ a and clinical¹⁵ studies suggest that activation of the naso-lacrimal reflex via intranasal stimulation also stimulates secretion of lipid and mucin.

Clinical studies have found that TrueTear® has a favorable safety profile, with mild self-limited nasal discomfort being the most frequent side effect of treatment.⁸ With regards to efficacy, a randomized, placebo controlled study of 48 individuals found that individuals randomized to active neurostimulation had higher Schirmer scores (mean 25.3 mm±10.7) compared to the sham control group (mean 9.2 mm±7.3, p-value<0.0001). In a follow-up open label study of 97 individuals, TrueTear® was applied 1.7±1.5 times per day over a 6 month period. At 6 months, TrueTear® was again found to increase tear production at the time of stimulation. Interestingly, while tear production consistently increased in the studies, improvement in dry eye symptoms was more variable and occurred in a minority of individuals.⁸ Another open label study evaluated the effect of TrueTear®, used 4 times daily (or more), on signs of DE. In 40 individuals with mild to severe DE, a reduction in mean corneal and conjunctival staining at 6 months was noted compared to baseline (3.0±1.0 vs 3.8±0.9, p=0.118 and 2.9±0.6 vs 4.4±0.4, p=0.002, respectively).¹⁶

Despite the demonstrated effectiveness of intranasal neurostimulation in stimulating tear production and improving corneal staining, there is limited literature regarding the efficacy of TrueTear® on symptom amelioration. Furthermore, while clinical tests have been developed to assess various components of DE (osmolarity, inflammation), limited tests are available to evaluate the nerve function. Described herein are results from studies carried out (1) to evaluate symptomatic improvement after 1 session of TrueTear® and assess whether improvement varied by DE sub-type and (2) to determine if change in tear production after stimulation correlated with other metrics of nerve dysfunction (e.g. neuropathic-like symptom profile″).

Methods of Treatment

Disclosed herein, are methods of treating ocular pain in a subject. Also, disclosed herein, are methods of reducing ocular pain in a subject. Further, disclosed herein, are methods of reducing ocular burning and/or ocular stinging in a subject. Also, disclosed herein, are methods of reducing or ameliorating one or more signs, symptoms, causes or effects of ocular pain in a subject. In some aspects, ocular pain can be described as a symptom or an indication of an underlying condition, disease or disorder. Ocular pain itself can be considered a diagnosis or a condition. In some aspects, the methods can comprise stimulating nasal tissue of the subject. In exemplary aspects, the subject does not have or exhibit low tear volume, which can correspond to a Schirmer score of less than 10 mm (under a broader definition) or less than 5 mm (under a narrower definition). For example, in some aspects, prior to treatment or reduction of ocular pain in the subject, the subject can have a Schirmer score of greater than or equal to 5 mm. Optionally, in these aspects, prior to treatment or reduction of ocular pain in the subject, the subject can have a Schirmer score of greater than or equal to 10 mm. In still other aspects, prior to treatment or reduction of ocular pain in the subject, the subject can have a Schirmer score of greater than or equal to 12 mm or greater than or equal to 15 mm or greater than or equal to 20 mm. In some aspects, the subject does not have dry eye disease. For example, it is contemplated that the subject can have ocular pain that is not caused by dry eye disease. As another example, the subject can have ocular pain that is not associated with other symptoms of dry eye disease as described herein.

Disclosed herein, are methods of treating a subject with ocular pain. The pain can be any ocular pain or associated with any condition or disorder. In some aspects, the ocular pain can be orofacial pain, macular degeneration, glaucoma, cataracts, optic neuritis, corneal disorders, corneal abrasions, iritis, uveitis, sinusitis, cluster headache, migraine, corneal ulcer, multiple sclerosis, blepharitis, meibomitis gland dysfunction, an autoimmune disease and diabetic retinopathy. In some aspects, the subject has been diagnosed with a condition or disorder associated with ocular pain prior to the administering step. In some aspects, ocular pain can be described as a symptom or an indication of an underlying condition, disease or disorder. Ocular pain itself can be considered a diagnosis or a condition.

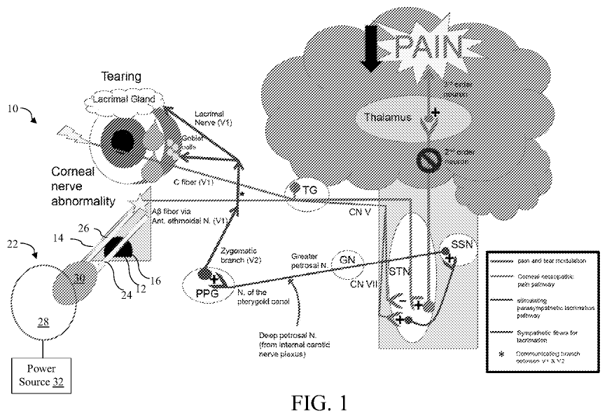

In some aspects, the stimulation system can be used to stimulate a subject's nasal tissue. Examples of said simulation systems include but are not limited to handheld stimulators (e.g., the TrueTear® handheld stimulator, as described in detail in U.S. Pat. Nos. 8,996,137, 9,440,065, 9,770,583, 9,956,397, 9,687,652, and 10,155,108, each of which is incorporated herein by reference in its entirety). Generally, the systems or handheld stimulator may be configured to stimulate nasal or sinus tissue. The devices may be handheld or implantable. Referring to FIG. 1, in some aspects, the stimulation system or device can comprise a stimulator body and a stimulator probe. In some aspects, the stimulator probe can comprise one or more nasal insertion prongs. In some aspects, the stimulus delivered by the stimulator can be electrical, mechanical, thermal, chemical, light-based, or magnetic.

The stimulation system described herein can be formulated to treat or reduce pain associated with one or more eyes. In some aspects, the stimulation system can be used to stimulate the nasal or sinus tissue 12 of a subject 10. In some aspects, the method can comprise inserting a first nasal insertion prong 24 of a stimulator probe 22 into a first nostril 16 of a nose 14 of the subject 10. In some aspects, the method can comprise inserting a second nasal insertion prong 26 of the stimulator probe 22 into a second nostril 18 of the nose 14. In some aspects, the electrodes can be positioned adjacent to a septum 20 of the subject 10 (for example, with one electrode on each of two opposing sides of the septum 20). In some aspects, the stimulator probe(s) can be connected to a stimulator body 28. In some aspects, the stimulator body 28 can comprise a control subsystem 30 to control an electrical stimulus to be delivered to the subject via the stimulator probe. In exemplary aspects, the control subsystem 30 can comprise a user interface that permits user selection and/or modification of stimulation parameters. The stimulator probe 22 can be in communication with a power source 32. In some aspects, a stimulus can be delivered to activate a nerve or pathway. In some aspects, the stimulus can have a maximum amplitude between 10 μA and 100 mA. In some aspects, the amplitude of the stimulus can be about 1.5 mA. Optionally, each stimulus can have a duration ranging from 30 to 60 seconds. In some aspects, the duration can be from 1 second to 10 minutes. In some aspects, the duration can be 1 second, 5 seconds, 10 seconds, 15 seconds, 20 seconds, 25 seconds, 30 seconds, 35 seconds, 40 seconds, 45 seconds, 50 seconds, 55 seconds, 60 seconds, or any number in between, or any number within a range defined between any of the stated values. In some aspects, the duration can be 1 minute, 2 minutes, 3 minutes, 4 minutes, 5 minutes, 6 minutes, 7 minutes, 8 minutes, 9 minutes, 10 minutes, or any number in between, or any number within a range defined between any of the stated values. In some aspects, each stimulus can comprise a waveform having a frequency between 20 Hz and 80 Hz. In some aspects, the stimulation can be delivered as a series of stimuli separated by a separation period. Optionally, the number of stimuli within the series of stimuli can range from 2 to 100, from 2 to 20, or from 3 to 10. In exemplary aspects, the number of stimuli within the series of stimuli can be 2, 3, 4, 5, 6, 7, 8, 9, 10, 11, 12, 13, 14, 15, 16, 17, 18, 19, or 20 or more stimuli. Optionally, the separation periods within the series of stimuli can be consistent (i.e., the same separation period can be used between each pair of consecutive stimuli. Alternatively, it is contemplated that the separation periods within the series of stimuli can vary (increase or decrease). Optionally, the amplitude of each stimuli within the series of stimuli can be the same (for example, 1.5 mA). Alternatively, the amplitude can be varied within the series of stimuli.

In some aspects, the stimulation can be delivered once. In some aspects, the stimulation protocol can be delivered more than once, such as, for example and without limitation, two, three, four, or five or more times within a treatment period as further disclosed herein. In some aspects, the stimulus can be delivered in a bipolar configuration between the first and second electrodes.

In some aspects, therapeutic stimulation of nasal or sinus tissue can encompass prophylactic applications. Based on genetic testing and other prognostic methods, a physician in consultation with their patient can choose a prophylactic administration where the patient has a clinically determined predisposition or increased susceptibility (in some cases, a greatly increased susceptibility) to ocular pain.

The methods of stimulating nasal or sinus tissue as described herein can be formulated in a variety of combinations. The particular combination or frequency of delivering the stimulation can vary according to many factors, for example, the particular the cause and severity of the ocular pain.

The methods described herein can be applied to the subject (e.g., a human patient) in an amount (intensity and/or frequency and/or duration) sufficient to delay, reduce, or preferably prevent the onset of ocular pain. Accordingly, in some aspects, the subject can be a human patient. In therapeutic applications, the methods and/or compositions can be administered to a subject (e.g., a human patient) already with or diagnosed with ocular pain or a condition associated with ocular pain in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of the stimulation (e.g., a pharmaceutical composition) can be an amount that achieves a cure, but that outcome is only one among several that can be achieved. As noted, a therapeutically effective amount includes amounts that provide a treatment in which the onset or progression of the ocular pain or condition or disorder is delayed, hindered, or prevented, or the ocular pain or condition or disorder or a symptom of the ocular pain or condition or disorder is ameliorated. One or more of the symptoms can be less severe. Recovery can be accelerated in an individual who has been treated.

In some aspects, the method can further comprise identifying a subject in need of treatment. In some aspects, the identifying step can comprise detecting one or more signs or symptoms of ocular pain, ocular burning or ocular stinging. In some aspects, one or more signs or symptoms of ocular pain, ocular burning or ocular stinging can be selected from the group consisting of scratching, stinging, itching, burning, redness, inflammation, discharge, headache, light sensitivity, aching, visual disturbances, or tearing. Optionally, in exemplary aspects, the identification of a subject in need of treatment can comprise identifying first and second groups of one or more subjects within a population of subjects having ocular pain, ocular burning, or ocular stinging, with the first group of subjects having low tear volume and the second group of subjects not having low tear volume. For example, it is contemplated that the first group of subjects can have a Schirmer score of less than 10 mm (optionally, less than 5 mm), while the second group of subjects can have a Schirmer score of greater than 10 mm (optionally, greater than 12 mm). In these aspects, it is contemplated that the first group of subjects can receive stimulation according to a first stimulation protocol, while the second group of subjects can receive stimulation according to a second stimulation protocol that is different from the first stimulation protocol in at least one of (optionally, each of) stimulation duration, stimulation frequency, or stimulation amplitude. Optionally, the second stimulation protocol can be different from the first stimulation protocol in at least stimulation duration and stimulation amplitude.

In some aspects, the one or more signs, symptoms, causes or effects of ocular pain in a subject can be selected from the group consisting of impaired vision, burning sensation, redness, irritation, inflammation, engorged vasculature, anterior lid margin vascularization, zone A posterior lid margin vascularization, eyelid disorders, swelling, vital staining, Schirmer's score, or Meibomian gland obstruction.

In some aspects, the ocular pain, condition or disorder can be one or more of orofacial pain, macular degeneration, glaucoma, cataracts, optic neuritis, corneal disorders, corneal abrasions, iritis, uveitis, sinusitis, cluster headache, migraine, corneal ulcer, multiple sclerosis, blepharitis, meibomitis gland dysfunction, an autoimmune disease and diabetic retinopathy. In some aspects, the autoimmune disease can be Sjögren's, multiple sclerosis, rheumatoid arthritis, or psoriatic arthritis. In some aspects, in any of the methods of treating or reducing ocular pain, ocular burning, or ocular stinging described herein, the subject has been diagnosed with any of the conditions or any of the disorders disclosed herein prior to stimulating the nasal tissue of the subject.

In some aspects, the ocular pain in the subject can be reduced. In some aspects, the ocular pain in the subject can be reduced wherein the reduction is not associated with a change in tear volume compared to the tear volume in the subject before stimulation. Optionally, in these aspects, the tear volume measured after stimulation (or after a treatment period) can be the same or approximately the same (e.g., within about 5, 10, or 15%) as the tear volume in the subject before stimulation. Optionally, in these aspects, the tear volume of the subject can be measured using Schirmer's test as is known in the art and further disclosed herein. Alternatively, it is contemplated that the tear volume of the subject can be measured using the phenol red thread test (PRT) as is known in the art and further disclosed herein.

The methods described herein can be used to treat acute and/or chronic ocular pain or any etiology associated with orofacial pain, macular degeneration, glaucoma, cataracts, optic neuritis, corneal disorders, corneal abrasions, iritis, uveitis, sinusitis, cluster headache, migraine, corneal ulcer, multiple sclerosis, blepharitis, meibomitis gland dysfunction, an autoimmune disease and diabetic retinopathy. In some aspects, the autoimmune disease can be Sjögren's, multiple sclerosis, rheumatoid arthritis, or psoriatic arthritis.

The stimulation described herein can be formulated to include a therapeutically effective amount (e.g., frequency and intensity). In some aspects, stimulation can be delivered to a subject at least once daily during a treatment period comprising at least one day to reduce ocular pain.

The therapeutically effective amount or stimulation used in the methods as disclosed herein applied to mammals (e.g., humans) can be determined by one of ordinary skill in the art with consideration of individual differences in age, weight, sex, other drugs administered and the judgment of the attending clinician. Variations in the needed amount and/or frequency and/or duration of stimulation may be expected. Variations in stimulation levels can be adjusted using standard empirical routes for optimization. The particular stimulation frequency and duration to be administered to the subject will depend on a variety of considerations (e.g., the severity of the ocular pain symptoms), the age and physical characteristics of the subject and other considerations known to those of ordinary skill in the art. Stimulation frequency and duration can be established using clinical approaches known to one of ordinary skill in the art.

The duration of treatment provided herein can be any length of time from as short as one day to as long as the life span of the host (e.g., many years). For example, the stimulation can be delivered once a day, once a week (for, for example, 4 weeks to many months or years); once a month (for, for example, three to twelve months or for many years); or once a year for a period of 5 years, ten years, or longer. It is also noted that the frequency of treatment can be variable. For example, the stimulation can be administered once (or twice, three times, etc.) daily, weekly, monthly, or yearly. In some aspects, the stimulation can be delivered once daily. In some aspects, the stimulation can be delivered once daily during a treatment period. In some aspects, the stimulation can be delivered two or more times during a treatment period. In some aspects, the treatment period can be one day, two days, three days, four days, five days, six days, seven days or more. In some aspects, the treatment period can be one week, two weeks, three weeks or longer.

The stimulation described herein can be delivered in conjunction with other therapeutic modalities to a subject in need of therapy. The present stimulation can be given to prior to, simultaneously with or after treatment with other agents or regimes. For example, the stimulation disclosed herein can be administered in conjunction with standard therapies used to treat pain or conditions or disorders associated with pain including ocular pain. In some aspects, stimulation described herein can be administered or used together with one or more analgesics. Suitable analgesics include, but are not limited to acetaminophen and acetaminophen-containing compounds and nonsteroidal anti-inflammatory (NSAID) drugs and NSAID-containing compounds such as, for example, salicylates, propionic acid derivatives, acetic acid derivatives, enolic acid derivatives, anthranilic acid derivatives, selective COX-2 inhibitors, sulfonanilides and LOX inhibitors. In some aspects, stimulation described herein can be administered or used together with topical cyclosporine, topical interleukin-1, or a combination thereof. In some aspects, cyclosporine or interleukin-1 can be administered at a concentration of 2.5% to 5%.

Any of the compounds or compositions or methods described herein can be administered as a term “combination.” It is to be understood that, for example, a C3a receptor inhibitor can be provided to the subject in need, either prior to administration of a C5a receptor inhibitor, an anti-C5 antibody or a C5 (or a C5a) aptamer, concomitant with administration of said a C5a receptor inhibitor, an anti-C5 antibody or a C5 (or a C5a) aptamer (co-administration) or shortly thereafter. It is also to be understood that, for example, a C5a receptor inhibitor can be provided to the subject in need, either prior to administration of a C₃a receptor inhibitor, an anti-C3 antibody or a C3 (or a C3a) aptamer, concomitant with administration of said a C3a receptor inhibitor, an anti-C3 antibody or a C3 (or a C3a) aptamer (co-administration) or shortly thereafter.

### Exemplary Aspects

In view of the described appliance, method, and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A method for reducing ocular pain in a subject, the method comprising stimulating nasal tissue of the subject.

Aspect 2: The method of aspect 1, wherein the ocular pain in the subject is reduced and is not associated with a change in tear volume compared to the tear volume in the subject before stimulation.

Aspect 3: A method of reducing ocular burning and/or ocular stinging in a subject, the method comprising stimulating nasal tissue of the subject.

Aspect 4: A method of reducing or ameliorating one or more signs, symptoms, causes or effects of ocular pain in a subject, the method comprising stimulating nasal tissue of the subject.

Aspect 5: The method of aspect 4, wherein the one or more signs, symptoms causes, or effects of ocular pain are burning, stinging, aching, scratching, itching, redness, inflammation, discharge, headache, light sensitivity, visual disturbances, tearing, or a combination thereof.

Aspect 6: The method of aspect 4, wherein the one or more signs, symptoms, causes or effects are selected from the group consisting of impaired vision, burning sensation, redness, irritation, inflammation, engorged vasculature, anterior lid margin vascularization, zone A posterior lid margin vascularization, eyelid disorders, swelling, vital staining, Schirmer's score, or Meibomian gland obstruction.

Aspect 7: A method for treating ocular pain in a subject, the method comprising stimulating nasal tissue of the subject.

Aspect 8: The method of aspect 7, wherein the ocular pain in the subject is reduced and is not associated with a change in tear volume compared to the tear volume in the subject before stimulation.

Aspect 9: The method of any of the preceding aspects, further comprising identifying the subject in need of treatment.

Aspect 10: The method of aspect 9, wherein the identifying step comprises detecting a sign or symptom selected from the group consisting of scratching, stinging, itching, burning, redness, inflammation, discharge, headache, light sensitivity, aching, visual disturbances, or tearing.

Aspect 11: The method of aspect 9 or aspect 10, wherein subject does not have low tear volume.

Aspect 12: The method of aspect 11, wherein the subject is a human having a Schirmer's score of greater than 10 mm before stimulation of the nasal tissue of the subject.

Aspect 13: The method of aspect 12, wherein the subject does not have dry eye disease.

Aspect 14: The method of any of the preceding aspects, wherein the subject has a condition that comprises one or more of or has been diagnosed with one or more of: orofacial pain, macular degeneration, glaucoma, cataracts, optic neuritis, corneal disorders, corneal abrasions, iritis, uveitis, sinusitis, cluster headache, migraine, corneal ulcer, multiple sclerosis, blepharitis, meibomitis gland dysfunction, an autoimmune disease and diabetic retinopathy.

Aspect 15: The method of aspect 14, wherein the autoimmune disease is Sjögren's, multiple sclerosis, rheumatoid arthritis, or psoriatic arthritis.

Aspect 16: The method of any of the preceding aspects, further comprising administering topical cyclosporine, topical interleukin-1, or a combination thereof.

Aspect 17: The method of aspect 16, wherein cyclosporine or interleukin-1 are administered at a concentration of 2.5% to 5%.

Aspect 18: The method of any one of the preceding aspects, wherein a first nasal insertion prong of a stimulator probe having a first electrode is inserted into a first nostril of a nose of the subject and a second nasal insertion prong of the stimulator probe having a second electrode is inserted into a second nostril of the nose, such that the first and second electrodes are positioned adjacent to a septum of the subject, wherein the stimulator probe is connected to a stimulator body, and wherein the stimulator body comprises a control subsystem to control an electrical stimulus to be delivered to the subject via the stimulator probe.

Aspect 19: The method of aspect 18, wherein the method further comprises: delivering the stimulus to activate a nerve, wherein the stimulus has a maximum amplitude between 10 pA and 100 mA and is delivered in a bipolar configuration between the first and second electrodes.

Aspect 20: The method of aspect 18 or aspect 19, wherein the stimulus comprises a waveform having a frequency between 20 Hz and 80 Hz.

Aspect 21: The method of any one of aspects 18-20, wherein the stimulus is delivered once daily during a treatment period.

Aspect 22: The method of any one of aspects 18-21, wherein the stimulus is delivered at an amplitude of about 1.5 mA.

Aspect 23: The method of any one of aspects 18-22, wherein the stimulus is delivered as a series of stimuli spaced 30 to 60 seconds apart.

### EXAMPLES

### Example 1: Effect of Noninvasive Intranasal Neurostimulation on Tear Volume, Dryness, and Ocular Pain by Dry Eye Sub-Type

Abstract

The purpose of the study described herein was to evaluate the effect of nasal-stimulation with TrueTear® on ocular pain and tear volume by dry eye (DE) sub-type.

Methods: Retrospective study of individuals with a variety of DE symptoms and signs. The individuals underwent a comprehensive ocular surface examination followed by one session of intranasal neurostimulation with TrueTear®. Outcome measures included objective change in tear volume measured via phenol red thread test (PRT) and subjective improvements in sensations of dryness and ocular pain measured on a 0-10 Numerical Rating Scale (NRS) scale.

Results: Seventy five of 86 individuals had a successful intranasal neurostimulation trial. Mean age of the 75 individuals was 59±13 years old; with a male majority (73.3%). Tear volume increased after stimulation by a mean of 13.40±8.00 mm, p<0.0005, while intensities of dryness and ocular pain lessened (mean reduction −2.85±2.79 for dryness and −1.48±2.41 for pain, p<0.0005 for both). In a multivariable model, the greatest predicator for increased tear volume was baseline tear volume (standardized beta (β)=−0.501, p=0.0005), followed by autoimmune disease (β=−0.355, p=0.001), i.e. individuals with initial lower tear volume and no autoimmune disease experienced greater increases in tear volume after one TrueTear® session than their counterparts. The strongest predictors for reduction in dryness and pain scores were baseline dryness and pain scores, i.e. lower scores at baseline correlated with greater improvement in scores after one TrueTear® session. Sixty-five percent of individuals rated the machine as easy to use and 56% indicated that would use the device at home. No complications related to nasal stimulation were noted.

Conclusion: Intranasal neurostimulation increased tear volume and decreased sensation of dryness and ocular pain after one use. Individuals with lower intensities of dryness and ocular pain prior to stimulation reported greater improvements in scores after stimulation.

Methods

Study Population: 86 individuals underwent a standardized ocular surface examination and underwent 1 session of TrueTear® stimulation. Inclusion criteria included individuals with a wide range of DE symptoms and signs. Individuals were excluded from participation if they presented with an active external ocular process, had any contraindication to neurostimulation (pacemaker, implanted or wearable defibrillator, or other electronic device in the head or neck), a known hypersensitivity to the hydrogel device material, chronic or recurrent nosebleeds, bleeding disorder, or a history of nasal or sinus surgery. Of the 86 individuals, 75 had a successful intranasal neurostimulation session, defined as the ability to insert the tips of the applicator to the appropriate location and induce a tearing response.

Measures: Demographic information (sex, age, race, and ethnicity), smoking history, past ocular and medical history, and medication information was collected for each participant.

Dry eye symptoms questionnaires: The participants filled out standardized questionnaires regarding dry eye symptoms, including the Ocular Surface Disease Index (OSDI, range 0-100)¹⁸ and the DEQS (range 0-22).¹⁹

Pain questionnaires: “Burning intensity”, “sensitivity to light”, “sensitivity to wind” and “sensitivity to contact with hot/cold” were assessed using a 0-10 numerical rating scale (NRS) over a 24 hour recall. A similar 0-10 scale was used to assess current “ocular pain intensity” and “dryness intensity” prior to nasal stimulation and immediately after.

Dry eye signs: Patients underwent a tear film assessment that included measurement of, in the order performed: (1) Phenol red thread test (PRT) (Zone Quick; Menicon, Nagoya, Japan) (right eye); (2) InflammaDry (Quidel, San Diego), graded as none, mild, moderate, severe based on the color of the pink line (both eyes); (3) anterior blepharitis, graded as 0=none; 1=mild; 2=moderate; 3=severe; (4) eyelid vascularity, graded as 0=none; 1=mild; 2=moderate; 3=severe engorgement; (5) meibomian gland inspissation, graded as 0=none; 1=mild; 2=moderate; 3=severe; (6) tear breakup time (TBUT), average of 3 measures per eye; (7) corneal staining graded to the NEI scale); and (8) Conjunctivochalasis, graded as absent or present in each area of the lower eyelid (temporal, central, nasal). The TrueTear® nasal stimulation session was then performed and PRT was re-measured from the right eye 15 seconds after the applicator was removed. The ocular exam then continued with a drop of anesthetic (proparicaine 1%) being placed in both eyes and (9) the presence of persistent ocular pain after anesthesia was assessed as yes or no; (10) Schirmer test with anesthesia graded as mm of wetting after 5 minutes; and (11) meibum quality, graded as 0=clear; 1=cloudy; 2=granular; 3=toothpaste; 4=no meibum extracted.

Intranasal neurostimulation protocol: The subjects placed the applicator into both nostrils simultaneously at a 45 degree angle. The applicator was advanced as far as comfortable with the goal of the end of the applicator reaching the lower nose. Stimulation intensity was set at level 2 (1.5 mA). The top of the applicator was repositioned along the inside surface of the nose to achieve the desired stimulation for 30-60 seconds. This test was done prior to topical anesthetic placement and Schirmer score assessment. Two minutes after completion of the session, individuals were asked to rate their pain and dryness and subjectively assess the device.

Main outcome measures: (1) Change in tear volume in the right eye measured via PRT (post—pre-stimulation). (2) Change in dryness and ocular pain via a 0-10 NRS scale (post—pre-stimulation). (3) Ease of use assessed via the question “How easy is it to use the device?” Responses included 0=difficult, 1=slightly difficult, 2=neutral, 3=easy, 4=very easy. (4) Qualitative assessment via the question “How do your eyes feel after using the device?” The response was described as 0=worse, 1=no change, 2=slightly better, 3=much better. (5) Likelihood of using device at home via the question “How often will you use the device at home?” Responses included 0=never, 1=rarely, 2=neutral, 3=sometimes, 4=frequently.

Statistical Analysis: Statistical analyses were performed using SPSS 22.0 (SPSS Inc, Chicago, Ill.) statistical package. Descriptive statistics were used to summarize patient demographic and clinical information. Means and standard deviations (SD) are expressed as mean±SD. Normality of distributions was assessed using the Kolmogorov-Smirnov (K-S) test statistic. Differences post-pre stimulation were assessed using paired t test comparisons. Correlation coefficients (Pearson r) were calculated between change in symptoms and tear volume and continuous variable (e.g. age, baseline dry eye symptoms). Comparisons of means with student t test were applied for nominal variables (e.g. gender, race). Multivariable forward step wise linear regression analyses were used to evaluate which baseline factors affected response to treatment (i.e. change in tear volume, dryness, and pain). The reported p values are two-tailed, and p<0.05 was considered statistically significant.

Results

Study Population: Overall, 86 individuals were seen and 75 had a successful intranasal neurostimulation trial. Mean age of the 75 individuals was 59±13 years old, with a male majority (73.3%) (Table 1). Nineteen of 75 patients had autoimmune diseases including 13 with Sjögren's, 5 with rheumatoid arthritis and 2 with psoriatic arthritis.

TABLE 1
Demographic and clinical data of the study population as they relate to change in
tear volume and ocular dryness and pain after 1 nasostimulation session, N = 75.

| | Descriptive | | ΔTear volume | ΔDryness | ΔOcular pain |
|---|---|---|---|---|---|
| Demographics | | | | | |
| Age years; mean ± | 59 ± 13 | (31-88) | r = −0.06; | r = −0.08; | r = −0.30; |
| SD (range) | | | p = 0.61 | p = 0.51 | p = 0.01 |
| Gender, % male (n) | 73.3% | (55) | M 14.18 ± 7.70 | −3.07 ± 2.66 | −1.53 ± 2.50 |
| | | | F 11.25 ± 8.66 | −2.25 ± 3.11 | −1.35 ± 2.18 |
| | | | p = 0.16 | p = 0.26 | p = 0.78 |
| Race, % white (n) | 50.7% | (38) | W = 12.84 ± 7.82 | −2.92 ± 3.03 | −1.79 ± 2.93 |
| | | | B = 12.74 ± 7.12 | −2.78 ± 2.49 | −1.26 ± 1.51 |
| | | | p = 0.96 | p = 0.85 | p = 0.43 |
| Ethnicity, % Hispanic | 33.3% | (25) | H 14.76 ± 8.68 | −3.24 ± 2.50 | −1.32 ± 1.44 |
| (H) (n) | | | NH 12.92 ± 7.70 | −2.75 ± 2.96 | −1.63 ± 2.82 |
| | | | p = 0.61 | p = 0.48 | p = 0.61 |
| Current smoker, % | 16.0% | (12) | Y 12.83 ± 6.34 | −3.17 ± 2.62 | −1.42 ± 1.50 |
| (n) | | | N 13.30 ± 8.64 | −2.62 ± 2.84 | −1.39 ± 2.40 |
| | | | p = 0.86 | p = 0.55 | p = 0.97 |
| Co-Morbidities | % (n) | | | | |
| Diabetes | 13.3% | (10) | Y 11.60 ± 5.76 | −2.50 ± 2.46 | −0.90 ± 1.45 |
| | | | N 13.78 ± 8.34 | −2.76 ± 2.92 | −1.71 ± 2.51 |
| | | | p = 0.43 | p = 0.79 | p = 0.33 |
| Hypertension | 36.0% | (27) | Y 12.18 ± 7.02 | −3.56 ± 2.55 | −1.70 ± 2.88 |
| | | | N 14.02 ± 8.59 | −2.51 ± 2.87 | −1.34 ± 2.14 |
| | | | p = 0.35 | p = 0.12 | p = 0.54 |
| Depression | 45.3% | (34) | Y 13.41 ± 8.04 | −2.85 ± 2.59 | −1.56 ± 2.13 |
| | | | N 13.39 ± 8.08 | −2.85 ± 2.97 | −1.41 ± 2.63 |
| | | | p = 0.99 | p = 0.99 | p = 0.80 |
| PTSD | 30.7% | (23) | Y 14.30 ± 8.32 | −2.87 ± 3.18 | −1.48 ± 1.38 |
| | | | N 13.00 ± 7.91 | −2.85 ± 2.63 | −1.48 ± 2.75 |
| | | | p = 0.52 | p = 0.97 | p = 0.99 |
| Traumatic brain injury | 13.3% | (10) | Y 17.60 ± 11.66 | −2.60 ± 2.12 | −0.40 ± 1.84 |
| | | | N 12.64 ± 7.19 | −2.86 ± 2.90 | −1.63 ± 2.47 |
| | | | p = 0.069 | p = 0.79 | p = 0.14 |
| Sleep Apnea | 41.3% | (31) | Y 13.84 ± 8.31 | −3.06 ± 2.57 | −1.11 ± 1.89 |
| | | | N 13.00 ± 7.93 | −2.77 ± 2.96 | −1.74 ± 2.73 |
| | | | p = 0.66 | p = 0.65 | p = 0.21 |
| Hypercholesterolemia | 26.7% | (20) | Y 14.15 ± 7.15 | −3.85 ± 3.33 | −2.35 ± 2.60 |
| | | | N 13.10 ± 8.41 | −2.63 ± 2.50 | −1.15 ± 2.33 |
| | | | p = 0.62 | p = 0.10 | p = 0.06 |
| Autoimmune disease | 25.3% | (19) | Y 10.11 ± 7.27 | −3.37 ± 3.58 | −1.68 ± 1.77 |
| | | | N 14.52 ± 7.99 | −2.68 ± 2.48 | −1.41 ± 2.60 |
| | | | p = 0.04 | p = 0.36 | p = 0.67 |
| Sjögren's disease | 17.3% | (13) | Y 11.38 ± 7.47 | −3.38 ± 3.59 | −2.15 ± 1.81 |
| | | | N 14.30 ± 8.18 | −2.55 ± 2.52 | −1.36 ± 2.59 |
| | | | p = 0.24 | p = 0.33 | p = 0.30 |
| Eye drops | % (n) | | | | |
| Artificial tears | 77.3% | (58) | Y 13.41 ± 8.12 | −2.97 ± 2.91 | −1.53 ± 2.43 |
| | | | N 13.35 ± 8.83 | −2.47 ± 2.35 | −1.29 ± 2.39 |
| | | | p = 0.98 | p = 0.52 | p = 0.72 |
| Fluorometholone | 10.7% | (8) | Y 11.00 ± 7.95 | −3.13 ± 2.42 | −0.50 ± 1.77 |
| 0.1% | | | N 13.83 ± 7.99 | −2.76 ± 2.82 | −1.56 ± 2.46 |
| | | | p = 0.35 | p = 0.73 | p = 0.24 |
| Cyclosporine 0.05% | 30.7% | (23) | Y 13.61 ± 8.25 | −3.13 ± 2.78 | −0.74 ± 1.82 |
| | | | N 13.49 ± 7.94 | −2.65 ± 2.77 | −1.76 ± 2.58 |
| | | | p = 0.95 | p = 0.49 | p = 0.09 |
| Lifitegrast | 8.0% | (6) | Y 15.00 ± 14.27 | −0.83 ± 3.87 | −1.50 ± 3.99 |
| | | | N 13.40 ± 7.35 | −2.97 ± 2.61 | −1.44 ± 2.26 |
| | | | p = 0.64 | p = 0.07 | p = 0.96 |
| Autologous Serum | 9.3% | (7) | Y 9.00 ± 10.50 | −5.00 ± 2.71 | −2.71 ± 4.15 |
| Tears | | | N 14.00 ± 7.62 | −2.57 ± 2.69 | −1.31 ± 2.15 |
| | | | p = 0.12 | p = 0.03 | p = 0.14 |
| Eye symptoms | Mean ± SD (range) | | | | |
| Eye dryness right | 5.2 ± 2.6 | (0-10) | r = 0.02; | r = −0.30; | r = −0.25; |
| before stimulation | | | p = 0.89 | p = 0.01 | p = 0.03 |
| (0-10) | | | | | |
| Eye pain right right | 4.04 ± 2.8 | (0-10) | r = −0.08; | r = −0.17; | r = −0.55; |
| before simulation | | | p = 0.50 | p = 0.15 | p = 0.0005 |
| (0-10) | | | | | |
| DEQ5 (0-22) | 14.9 ± 3.8 | (4-22) | r = 0.07; | r = 0.15; | r = 0.003; |
| | | | p = 0.56 | p = 0.21 | p = 0.98 |
| OSDI (0-100) | 50.7 ± 22.1 | (0-100) | r = −0.08; | r = 0.009; | r = −0.07; |
| | | | p = 0.50 | p = 0.94 | p = 0.57 |
| Burning, avg 24 | 4.1 ± 3.1 | (0-10) | r = −0.05; | r = 0.18; | r = 0.04; |
| hours (0-10) | | | p = 0.70 | p = 0.11 | p = 0.71 |
| Pain evoked by wind, | 4.8 ± 3.0 | (0-10) | r = −0.18; | r = 0.08; | r = −0.05; |
| avg 24 hours (0-10) | | | p = 0.12 | p = 0.50 | p = 0.66 |
| Pain evoked by light, | 5.1 ± 3.3 | (0-10) | r = −0.07; | r = 0.04; | r = −0.08; |
| avg 24 hours (0-10) | | | p = 0.53 | p = 0.76 | p = 0.50 |
| Pain evoked by cold | 4.5 ± 3.3 | (0-10) | r = −0.02; | r = 0.11; | r = −0.01; |
| or hot, avg 24 hours | | | p = 0.86 | p = 0.34 | p = 0.96 |
| (0-10) | | | | | |
| Dry Eye Signs | Mean ± SD (range) | | | | |
| Pre-stimulation PRT | 16.01 ± 9.51 | | r = −0.42; | r = 0.12; | r = 0.01; |
| (2-42) | | | p < 0.0005 | p = 0.35 | p = 0.93 |
| InflammaDry (0-3) | 1.5 ± 1.0 | (0-3) | r = −0.16; | r = 0.15; | r = 0.08; |
| | | | p = 0.161 | p = 0.209 | p = 0.506 |
| Anterior blepharitis | 0.6 ± 1.0 | (0-3) | r = −0.16; | r = −0.10; | r = −0.13; |
| (0-3) | | | p = 0.18 | p = 0.40 | p = 0.25 |
| MG inspissation (0-3) | 0.8 ± 0.8 | (0-3) | r = −0.11; | r = −0.05; | r = −0.23; |
| | | | p = 0.34 | p = 0.66 | p = 0.05 |
| Eyelid vascularity | 1.2 ± 1.1 | (0-3) | r = −0.17; | r = −0.10; | r = −0.28; |
| (0-3) | | | p = 0.15 | p = 0.39 | p = 0.01 |
| TBUT (seconds) | 5.7 ± 3.6 | (2-19) | r = 0.001; | r = 0.03; | r = −0.02; |
| | | | p = 0.99 | p = 0.82 | p = 0.88 |
| Cornea staining | 3.7 ± 3.4 | (0-13) | r = −0.08; | r = 0.04; | r = −0.12; |
| (0-15) | | | p = 0.51 | p = 0.73 | p = 0.31 |
| Temporal | 65.3% | (49) | Y 13.36 ± 7.94 | −2.91 ± 2.94 | −1.96 ± 2.45 |
| conjunctiochalasis | | | N 13.11 ± 8.45 | −2.77 ± 2.66 | −0.58 ± 2.18 |
| | | | p = 0.90 | p = 0.83 | p = 0.02 |
| Nasal | 29.3% | (22) | Y 12.86 ± 9.45 | −2.50 ± 3.20 | −2.00 ± 2.96 |
| conjunctiochalasis | | | N 13.62 ± 7.41 | −3.00 ± 2.62 | −1.26 ± 2.13 |
| | | | p = 0.71 | p = 0.48 | p = 0.23 |
| Persistent pain after | 53.3% | (40) | Y 13.40 ± 8.31 | −2.35 ± 2.92 | −1.10 ± 2.29 |
| anesthesia, % (n) | | | N 13.40 ± 7.83 | −3.43 ± 2.55 | −1.91 ± 2.49 |
| | | | p = 1.00 | p = 0.10 | p = 0.15 |
| Schirmer's test | 11.2 ± 7.6 | (2-35) | r = −0.01; | r = −0.17; | r = −0.02; |
| (mm wetting) | | | p = 0.93 | p = 0.14 | p = 0.85 |
| Meibum quality (0-4) | 2.1 ± 1.1 | (0-4) | r = −0.07; | r = 0.003; | r = −0.21; |
| | | | p = 0.56 | p = 0.98 | p = 0.07 |

SD = Standard deviation,
PTSD = post-traumatic stress disorder;
n = number;
avg = average,
PRT = phenol red thread;
rho = Spearman's rank-order;
r = Pearson's correlation coefficients;
F = female;
M = male;
W = white;
B = black;
H = Hispanic;
NH = non-Hispanic;
Y = yes;
N = no;
DEQ5 = dry eye questionnaire 5;
OSDI = ocular Surface disease index;
MMP-9 = matrix metalloproteinase 9,
MG = meibomian gland,
TBUT = Tear break up time

Change in tear volume: Tear volume, as measured via PRT, increased after one stimulation session (mean change 13.40±8.00 mm, p<0.0005). Some baseline measures correlated with change in PRT. Specifically, patients with autoimmune disease had less of an increase in volume compared to non-autoimmune (10.11±7.27; 14.52±7.99, p=0.04) (Table 1). Baseline tear volume was negatively correlated with change in volume, implying that those with lower baseline tear volumes had a greater increase in volume post-pre stimulation (r=−0.42). A multivariable forward step wise linear regression model was performed including demographics and the factors that significantly predicted change in PRT scores on univariable analysis. Of these, baseline PRT value (standardized beta (β)=−0.501, p=0.0005), and autoimmune disease (β=−0.355, p=0.001) remained in the model.

Change in ocular dryness and pain: Overall both ocular dryness and pain intensity decreased after neurostimulation (mean change −2.85±2.79 for dryness and −1.48±2.41 for pain, p<0.0005 for both). Nineteen (25.3%) individuals reported no change in dryness after the session and 2 (2.6%) reported worse dryness. In a similar manner, 24 (32.0%) individuals reported no change in pain and 5 (6.7%) more severe pain after stimulation. Several factors predicted a larger improvement in dryness intensity including lower pre-treatment dryness intensity (r=−0.30) and lower Schirmer score (r=−0.17). Factors that predicted a larger improvement in ocular pain intensity including younger age (r=−0.30), lower dryness scores prior to stimulation (r=−0.25), lower eye pain on average across a one week recall (r=−0.30), and more healthy eyelid parameters (r=−0.23 for meibomian gland inspissation and r=−0.28 for eyelid vascularity). A multivariable forward step wise linear regression model was performed including demographics and the factors that significantly predicted change in dryness scores on univariable analysis. Of these, baseline ocular dryness score (β=−0.455, p<0.0005) remained in the model. Regarding ocular pain, baseline ocular pain (β=−0.569, p<0.0005) and age (β=−0.348, p<0.0005) were the significantly predictors of change in ocular pain scores.

Subjective responses: The device was overall well accepted with 65% rating the machine as easy to use (rating of “easy” or “very easy”) while 16% felt it was “difficult”. Overall, 81% of individuals states that their eyes felt “slightly” or “much” better after treatment while 4% felt worse. No complications related to the nasal stimulation were noted. 56% reported that they would use the device “frequently” at home.

### Discussion

First it was determined whether there was a differential symptomatic response to 1 session of TrueTear® by DE subtype. The results show that an overall reduction in subjective symptoms of dryness and pain was observed for the entire group tested. However, individuals with lower intensity of dryness and pain pre-stimulation had a greater improvement in symptoms after stimulation. In addition, younger individuals experienced greater pain relief compared to older individuals. These findings suggest that other mechanisms, beyond tear volume, contribute to dryness and pain in individuals with high pain intensity. It was also found that individuals with less healthy anatomy (e.g., inspissation, vascularity, conjunctivochalasis) had less of an improvement in pain after nasostimulation, likely based on the same rationale as above.

Next, it was determined if a change in tear volume after 1 intranasal neurostimulation could be used as a surrogate measure of naso-lacrimal arc reactivity. It was tested whether individuals with symptoms consistent with neuropathic pain (e.g., burning, evoked pain to wind and light, and persistent pain after anesthesia) would demonstrate a more robust change in PRT, reflective of a more sensitive naso-lacrimal arc. The data do not support this notion, however, as no baseline DE metrics, beyond tear volume, correlated with change in PRT. It is not surprising, however, that individuals with a systemic autoimmune disease had less of a volume increase than their counterparts without an autoimmune disease, as this subset of patients are more likely to have ATD due to lacrimal gland inflammation and fibrosis.²¹

With respect to tear stimulation, the results were similar to prior TrueTear® studies. The results showed a mean increase of 13.40±8.00 mm in tear volume (via PRT) in the population with a diverse range of DE symptoms and signs. In studies of 1 TrueTear® session in 48⁸ and 10 patients¹⁵ with DE, tear production via Schirmer increased by 16.1 mm⁸ and tear meniscus high via anterior segment optical coherence tomography (AS-OCT) increased by 43.1 μm,¹⁵ respectively. After 6 months of use, the ability of TrueTear® to increase tear production was again demonstrated with Schirmer change that ranged from 6.8¹⁶ to 9.4⁸ mm across different DEWS severity category levels.

TrueTear® is not the first device used to treat ocular disease with electrical stimulation. Prior to the development TrueTear®, the utility of peripheral nerve stimulation was demonstrated in accelerating corneal healing²², improving corneal sensitivity²³, and resolving postoperative ocular pain.^{24, 25} Electrical stimulation has also been evaluated in animal models both invasively, with electrodes implanted beneath the inferior lacrimal gland, and non-invasively with a monopolar electrode placed near the afferent ethmoid nerve.^{26, 27} Improved tear production was noted using both methods. Peripheral stimulation has also been used to treat multiple oral and facial pain conditions.²⁸⁻³²

Indeed, non-invasive peripheral stimulation of V1 branches of the trigeminal nerve improved light sensitivity and ocular pain in a pilot study of 14 individuals with sensations of dryness from presumed neuropathic mechanisms.³³ The study showed that the transcutaneous electrical stimulation (TENS) of the ocular region decreased ocular pain intensity by 2.62±0.68 (right eye) and 2.46±0.98 (left eye), which was comparable in the results disclosed herein (1.48±2.41) and others (2.96±0.59)¹⁶ by using intranasal neurostimulation. Using intranasal neurostimulation to relieve DE symptoms, including ocular pain, is based on the notion that the body's natural tear production can be upregulated by activation of the nasolacrimal reflex, a reflex that plays an important role in expelling foreign bodies or irritants by secreting tears. Ocular pain is also linked this trigeminal-thalamic-cortical pathway in which the neuroanatomical input of corneal sensation travels from nasociliary branch of V1 branch of the trigeminal nerve to the spinal trigeminal nuclear complex (Vi/Vc, Vc/C1). The second order neurons join the contralateral spinothalamic pathway to project to the thalamus and the third order neurons synapse to ascend to the somatosensory cortex and limbal system.³⁴ The anterior ethmoidal nerve in the nasal cavity is an extraconal branch of the nasociliary nerve, which is a branch of V1. Keeping the anatomy in mind, the reduction in ocular pain intensity after neurostimulation may be related to modulation of pain pathways along with improvements in tear volume and potentially, improvements in mucin and lipid health as well.

Based on the results from responses to one session of TrueTear® by DE subtype, it was found that overall, tear volume improved and ocular dryness and pain sensations decreased in those that completed a successful session. However, some DE sub-types had a better objective and subjective response, including those with a lower degree of dryness and ocular pain and those with lower baseline tear production.

### REFERENCES

1. Stapleton F, Alves M, Bunya V Y, et al. TFOS DEWS II Epidemiology Report. Ocul Surf 2017; 15(3):334-65.
2. Craig J P, Nichols K K, Akpek E K, et al. TFOS DEWS II Definition and Classification Report. Ocul Surf 2017; 15(3):276-83.
3. Jones L, Downie L E, Korb D, et al. TFOS DEWS II Management and Therapy Report. Ocul Surf 2017; 15(3):575-628.
4. Galor A, Moein H R, Lee C, et al. Neuropathic pain and dry eye. Ocul Surf 2017.
5. Aggarwal S, Kheirkhah A, Cavalcanti B M, et al. Autologous Serum Tears for Treatment of Photoallodynia in Patients with Corneal Neuropathy: Efficacy and Evaluation with In Vivo Confocal Microscopy. Ocul Surf 2015; 13(3):250-62.
6. Diel R J, Kroeger Z A, Levitt R C, et al. Botulinum Toxin A for the Treatment of Photophobia and Dry Eye. Ophthalmology 2018; 125(1):139-40.
7. Diel R J, Hwang J, Kroeger Z A, et al. Photophobia and sensations of dryness in patients with migraine occur independent of baseline tear volume and improve following botulinum toxin A injections. Br J Ophthalmol 2018.
8. https://allergan-web-cdn-prod.azureedge.net/actavis/actavis/media/allergan-pdf-documents/labeling/ifu truetearprofessional.pdf. Accessed Aug. 14, 2018.
9. Zilstorff-Pedersen K. Quantitative Measurements of the Nasolacrimal Reflex. Arch Otolaryngol 1965; 81:457-62.
10. Belmonte C, Nichols J J, Cox S M, et al. TFOS DEWS II pain and sensation report. Ocul Surf 2017; 15(3):404-37.
11. Ding C, Walcott B, Keyser K T. Sympathetic neural control of the mouse lacrimal gland. Invest Ophthalmol Vis Sci 2003; 44(4):1513-20.
12. Parod R J, Putney J W, Jr. An alpha-adrenergic receptor mechanism controlling potassium permeability in the rat lacrimal gland acinar cell. J Physiol 1978; 281:359-69.
13. Dartt D A, Rose P E, Dicker D M, et al. Alpha 1-adrenergic agonist-stimulated protein secretion in rat exorbital lacrimal gland acini. Exp Eye Res 1994; 58(4):423-9.
14. Brinton M, Kossler A L, Patel Z M, et al. Enhanced Tearing by Electrical Stimulation of the Anterior Ethmoid Nerve. Invest Ophthalmol Vis Sci 2017; 58(4):2341-8.
15. Gumus K, Schuetzle K L, Pflugfelder S C. Randomized Controlled Crossover Trial Comparing the Impact of Sham or Intranasal Tear Neurostimulation on Conjunctival Goblet Cell Degranulation. Am J Ophthalmol 2017; 177:159-68.
16. Friedman N J, Butron K, Robledo N, et al. A nonrandomized, open-label study to evaluate the effect of nasal stimulation on tear production in subjects with dry eye disease. Clin Ophthalmol 2016; 10:795-804.
17. Crane A M, Levitt R C, Felix E R, et al. Patients with more severe symptoms of neuropathic ocular pain report more frequent and severe chronic overlapping pain conditions and psychiatric disease. Br J Ophthalmol 2017; 101(2):227-31.
18. Schiffman R M, Christianson M D, Jacobsen G, et al. Reliability and validity of the Ocular Surface Disease Index. Arch Ophthalmol 2000; 118(5):615-21.
19. Chalmers R L, Begley C G, Caffery B. Validation of the 5-Item Dry Eye Questionnaire (DEQ-5): Discrimination across self-assessed severity and aqueous tear deficient dry eye diagnoses. Cont Lens Anterior Eye 2010; 33(2):55-60.
20. Wolffsohn J S, Arita R, Chalmers R, et al. TFOS DEWS II Diagnostic Methodology report. Ocul Surf 2017; 15(3):539-74.
21. Bron A J, de Paiva C S, Chauhan S K, et al. TFOS DEWS II pathophysiology report. Ocul Surf 2017; 15(3):438-510.
22. Ghaffarieh A, Ghaffarpasand F, Dehghankhalili M, et al. Effect of transcutaneous electrical stimulation on rabbit corneal epithelial cell migration. Cornea 2012; 31(5):559-63.
23. Ghaffariyeh A, Peyman A, Puyan S, et al. Evaluation of transcutaneous electrical simulation to improve recovery from corneal hypoesthesia after LASIK. Graefes Arch Clin Exp Ophthalmol 2009; 247(8):1133-8.
24. Ticho U, Olshwang D, Magora F. Relief of pain by subcutaneous electrical stimulation after ocular surgery. Am J Ophthalmol 1980; 89(6):803-8.
25. Whitacre M M. The effect of transcutaneous electrical nerve stimulation on ocular pain. Ophthalmic Surg 1991; 22(8):462-6.
26. Kossler A L, Wang J, Feuer W, Tse D T. Neurostimulation of the lacrimal nerve for enhanced tear production. Ophthalmic Plast Reconstr Surg 2015; 31(2):145-51.
27. Brinton M, Chung J L, Kossler A, et al. Electronic enhancement of tear secretion. J Neural Eng 2016; 13(1):016006.
28. Kasat V, Gupta A, Ladda R, et al. Transcutaneous electric nerve stimulation (TENS) in dentistry-A review. Journal of clinical and experimental dentistry 2014; 6(5):e562.
29. Johnson M D, Burchiel K J. Peripheral stimulation for treatment of trigeminal postherpetic neuralgia and trigeminal posttraumatic neuropathic pain: a pilot study. Neurosurgery 2004; 55(1):135-42.
30. Holsheimer J. Electrical stimulation of the trigeminal tract in chronic, intractable facial neuralgia. Archives of physiology and biochemistry 2001; 109(4):304-8.
31. Eriksson M B, Sjölund BH, Sundbarg G. Pain relief from peripheral conditioning stimulation in patients with chronic facial pain. Journal of neurosurgery 1984; 61(1): 149-55.
32. Hansson P, Ekblom A. Transcutaneous electrical nerve stimulation (TENS) as compared to placebo TENS for the relief of acute oro-facial pain. Pain 1983; 15(1):157-65.
33. Sivanesan E, Levitt R C, Sarantopoulos C D, et al. Noninvasive Electrical Stimulation for the Treatment of Chronic Ocular Pain and Photophobia. Neuromodulation 2018; 21(8):727-34.
34. Parra A, Gonzalez-Gonzalez O, Gallar J, Belmonte C. Tear fluid hyperosmolality increases nerve impulse activity of cold thermoreceptor endings of the cornea. Pain 2014; 155(8):1481-91.

### Example 2: Effect of Noninvasive Intranasal Neurostimulation on Tear Volume, Dryness, and Ocular Pain

Synopsis: One session of intranasal neurostimulation increased tear volume and reduced sensations of dryness and ocular pain, independently of each other.

Abstract

Purpose. To evaluate the effect of one TrueTear® session on change in tear volume and symptoms of dryness and ocular pain.

Methods. Retrospective interventional case-series of patients seen in a clinic. Seventy-five individuals underwent an ocular surface examination and one session of neurostimulation. Outcome measures included objective change in tear volume measured via phenol red test and subjective change in sensations of dryness and ocular pain measured on a 0-10 Numerical Rating Scale.

Results. Mean age of the 75 individuals was 59±13 years and the majority were male (73%). Intranasal neurostimulation increased tear volume (mean 13.40±8.00 mm, p<0.0005) and reduced intensities of dryness (mean −2.85±2.79, p<0.0005) and ocular pain (mean −1.48±2.41, p<0.0005 for both). However, these effects were independent of one another as change in symptom report did not correlate with change in tear volume (r=−0.13, p=0.25 for dryness, r=0.07, p=0.56 for pain). In a multivariable model, the strongest predictors for increased tear volume were lower baseline tear volume (standardized beta (β)=−0.50, p<0.0005) and absence of an autoimmune disease (β=−0.36, p=0.001), R²=0.30. The strongest predictors for reduced dryness and pain scores were lower baseline dryness and ocular pain scores. No complications related to neurostimulation were noted.

Conclusion. Intranasal neurostimulation increased tear volume and reduced intensities of dryness and ocular pain, independently of one another.

### Introduction

Dry eye (DE) is a common condition associated with significant ocular morbidity.[1] It is a multifactorial disease that can include tear film and ocular surface disturbances, such as aqueous and evaporative deficiency, and/or nerve dysfunction, which often overlap and interact. [2] These mechanisms result in symptoms which include ocular pain, described in terms of “dryness”, “burning,” and “aching,” and visual disturbances. Treatments for DE target tear health by increasing tear volume with artificial tears, decreasing inflammation with corticosteroids, cyclosporine, or lifitegrast, addressing Meibomian gland (MG) dysfunction and tear instability with lid hygiene and antibiotics, and blocking tear outflow with punctal closure.[3] Alpha 2 delta (a2.5) ligands (gabapentin and pregabalin)[4], autologous serum tears[5], and botulinum toxin injection[6 7] have been used in individuals with DE symptoms in the setting of presumed neuropathic mechanisms. Unfortunately, results are variable and some individuals have persistent DE symptoms and signs despite therapy.

Recently, a device was approved for the treatment of low tear volume which targets the neurophysiology of the lacrimal functional unit. TrueTear® (Allergan, San Diego, Calif.) stimulates the anterior ethmoidal nerve with adjustable pulses of energy, up to a maximum of 13 V or 5 mA at 30-60 Hz, which activates the nasolacrimal reflex. [8] The anterior ethmoidal nerve is an extraconal branch of the nasociliary nerve which in turn is a branch of ophthalmic division of the trigeminal nerve. Stimulation of this nerve activates the afferent limb of the nasolacrimal reflex. [9] The efferent limb of the reflex originates from the superior salivary nucleus. Signals then travel along the facial nerve, through the geniculate ganglion, the greater superficial petrosal nerve, and the nerve of the pterygoid canal to the pterygopalatine ganglion (PPG) and then via the zygomatic nerve to the lacrimal nerve and the lacrimal gland (FIG. 1). [10] While reflex aqueous tear secretion is predominantly regulated by this parasympathetic arc, sympathetic nerves from the superior cervical ganglion (SCG) also affect aqueous production and the composition of tear proteins and electrolytes.[11] While the innervation pattern of the MG (for lipids) and goblet cells (for mucin) are less well characterized[10], animal[12] and clinical[13] studies suggest that activation of the nasolacrimal reflex via intranasal stimulation also stimulates secretion of lipids and mucin.

Clinical studies have found a favorable safety profile for TrueTear®, with mild nose bleeds and self-limited nasal discomfort as the most frequent side effects.[8 14] With regard to efficacy, a randomized, placebo controlled study of 56 individuals found that individuals randomized to active treatment had higher post-stimulation Schirmer scores (mean 24.2 mm) compared to the sham group (mean 9.0 mm), p-value<0.001.[14] Subjects then used the device 4 to 8 times a day, and on repeat evaluation 3 months later, the active treatment group once again had a greater increase in tear production after stimulation compared to the sham group. Interestingly, while tear production consistently increased after stimulation in the studies[8 14 15], reduction in other DE symptoms was more variable. Overall, DE symptom scores decreased with time[8 14 15], but this did not occur uniformly, with 62% of subjects reporting better or somewhat better symptoms than before starting neurostimulation, 30% reporting no change, and 2% reporting worsening symptoms in one study. [8] Unlike information on tear production, there is a paucity of data on the effect of one TrueTear® session specifically on pain symptoms associated with DE. Furthermore, while point of care tests have been developed to assess the contributions of tear osmolarity and ocular surface inflammation on DE, few tests are available to evaluate corneal nerve status. As such, the study described herein: (1) quantified the change in symptoms of dryness and pain after one session of TrueTear® and assessed baseline demographic and ocular surface factors that impacted symptom reduction and (2) determined whether change in tear volume after a neurostimulation session could be used as a surrogate measure of nasolacrimal arc reactivity, testing whether individuals with neuropathic-like pain symptom profile[16] would have a more robust increase in tear volume than their counterparts without this symptom profile.

Methods

Study Population: 86 individuals underwent a standardized ocular surface examination and one session of TrueTear® stimulation. Individuals recruited from this clinic presented with a wide range of DE symptoms and signs. Individuals were excluded from participation if they presented with an active external ocular process, had any contraindication to neurostimulation (pacemaker, implanted or wearable defibrillator, or other electronic device in the head or neck), a known hypersensitivity to the hydrogel device material, chronic or recurrent nosebleeds, bleeding disorder, or a history of nasal or sinus surgery.

Data collection: Demographic information (sex, age, race, and ethnicity), smoking history, past ocular and medical history, and medication information was collected for each participant.

Dry eye symptoms: Participants filled out standardized questionnaires regarding DE symptoms, including the Ocular Surface Disease Index (OSDI, range 0-100)[17] and DEQS (range 0-22).[18] In addition, spontaneous and evoked pain were assessed by asking individuals to rate their “burning intensity”, “sensitivity to light”, “sensitivity to wind” and “sensitivity to contact with hot/cold” using a 0-10 Numerical Rating Scale (NRS) over a 24 hour recall.

Dry eye signs: Participants underwent an ocular surface examination that included the following measures, in the order performed: (1) Phenol red thread test (PRT) (Zone Quick; Menicon, Nagoya, Japan) (right eye) performed by inserting a 75 mm long phenol red coated thread into the lower fornix for 15 seconds. The thread was then removed and the length of the red line measured. Of note, normal tear volume is defined as >20 mm, borderline volume between 10 and 19 mm, and severe reduction as <10 mm.[19]; (2) Ocular surface inflammation qualitatively graded as none, mild, moderate, severe based on the intensity of the pink stripe (InflammaDry, Quidel, San Diego); (3) Eyelid metrics qualitatively graded at the slit lamp without touching the eyelids included anterior blepharitis (0-3), defined as the quantity of debris on the eyelashes, eyelid vascularity (0-3), and MG plugging (0-3), defined as the presence of white plugs at the opening of the MG [20]; (6) Tear breakup time (TBUT) recorded as the average of 3 measures per eye; (7) Corneal staining graded to the NEI scale[21]; and (8) Conjunctivochalasis graded as absent or present in each area of the lower eyelid (temporal, central, nasal).

Intranasal neurostimulation protocol: The TrueTear® session was then performed. First, intensity of dryness and ocular pain were assessed immediately prior to neurostimulation via a 0-10 NRS. Then, subjects placed the applicator into both nostrils simultaneously at a 45 degree angle. The applicator was advanced as far as comfortable with the goal of the end of the applicator reaching the opening of the nares. Stimulation intensity was set at level 2 (1.5 mA). The top of the applicator was repositioned along the inside surface of the nose to achieve the desired stimulation for 30-60 seconds and a gush of tears was noted by the observer. Two minutes after stimulation, subjects were again asked to rate the intensity of dryness and ocular pain. Tear volume (PRT) was assessed in the right eye. Individuals then rated the ease of device use via the question “How easy was it to use the device?” with choices being difficult, slightly difficult, neutral, easy, and very easy. Individuals were also asked “How do your eyes feel after using the device?” with choices being worse, no change, slightly better, and much better. Finally, individuals were asked “How often would you use the device at home?” with choices being never, rarely, sometimes, and frequently.

The ocular surface examination then commenced after anesthetic (proparacaine 1%) was placed in both eyes and after 1 minute, persistent ocular pain after anesthesia was assessed as yes or no and if present, graded on a 1-10 scale. Schirmer strips were then placed and mm of wetting after 5 minutes recorded; After pressing on the eyelid with 2 cotton tip applicators, Meibum quality was qualitatively graded as 0=clear; 1=cloudy; 2=granular; 3=toothpaste; 4=no Meibum extracted. [20]

DE categories: Individuals were classified into DE categories based on ocular surface findings prior to nasal stimulation. Aqueous tear deficiency (ATD) was defined as a PRT value of <10 mm in either eye and evaporative deficiency as a PRT value of ≥10 mm in both eyes and TBUT<5 seconds in either eye.

Main outcome measures: Main outcome measures included change in tear volume (post—pre-stimulation) and change in intensities of dryness and ocular pain (post—pre-stimulation). Furthermore, change in volume was correlated to metrics associated with nerve abnormalities (pain, burning, sensitivity to wind and light). [22 23]

Statistical Analysis: Statistical analyses were performed using SPSS 26.0 (IBM, Chicago, Ill.). Descriptive statistics were used to summarize patient demographic and clinical information. Normality of distributions was assessed using the Kolmogorov-Smirnov (K-S) test. Post versus pre stimulation differences were assessed using paired t test comparisons. Correlation coefficients (Pearson r) examined relationships between symptoms and tear volume changes with nasal stimulation and continuous variables (e.g. age). Student t tests were applied for nominal variables (e.g. gender, race). Multivariable forward step wise linear regression analyses were used to evaluate which baseline factors affected responses to stimulation. The reported p values are two-tailed, and p values<0.05 were considered statistically significant.

Results

Study Population: Overall, 86 individuals were seen and 75 had a successful intranasal neurostimulation trial, defined as the ability to insert the tips of the applicator to the appropriate location and induce a tearing response. Of the 11 individuals who did not have a successful trial, 3 had a contraindication to TrueTear® and 8 could not get the tips into an optimal position and a tearing response was not elicited. Mean age of the 75 individuals was 59±13 years old with a male majority (73.3%) (Table 2). Nineteen of 75 patients had an autoimmune disease, including 13 with Sjögrens syndrome, 5 with rheumatoid arthritis, and 2 with psoriatic arthritis. The majority of individuals were using one more DE therapies as indicated in Table 2.

TABLE 2
Demographic and clinical data of the study population as they relate to change in
tear volume, dryness, and ocular pain after one nasal stimulation session, N = 75

| | Descriptive | | ΔTear volume | ΔDryness | ΔOcular pain |
|---|---|---|---|---|---|
| Demographics | | | | | |
| Age years; mean ± SD | 59 ± 13 | (31-88) | r = −0.06; | r = −0.08; | r = −0.30; |
| (range) | | | p = 0.61 | p = 0.51 | p = 0.01 |
| Gender, % male (n) | 73.3% | (55) | M 14.18 ± 7.70 | −3.07 ± 2.66 | −1.53 ± 2.50 |
| | | | F 11.25 ± 8.66 | −2.25 ± 3.11 | −1.35 ± 2.18 |
| | | | p = 0.16 | p = 0.26 | p = 0.78 |
| Race, % white (n) | 50.7% | (38) | W = 12.84 ± 7.82 | −2.92 ± 3.03 | −1.79 ± 2.93 |
| | | | B = 12.74 ± 7.12 | −2.78 ± 2.49 | −1.26 ± 1.51 |
| | | | p = 0.96 | p = 0.85 | p = 0.43 |
| Ethnicity, % Hispanic | 33.3% | (25) | H 14.76 ± 8.68 | −3.24 ± 2.50 | −1.32 ± 1.44 |
| (H) (n) | | | NH 12.92 ± 7.70 | −2.75 ± 2.96 | −1.63 ± 2.82 |
| | | | p = 0.61 | p = 0.48 | p = 0.61 |
| Current smoker, % (n) | 16.0% | (12) | Y 12.83 ± 6.34 | −3.17 ± 2.62 | −1.42 ± 1.50 |
| | | | N 13.30 ± 8.64 | −2.62 ± 2.84 | −1.39 ± 2.40 |
| | | | p = 0.86 | p = 0.55 | p = 0.97 |
| Co-Morbidities, % (n) | | | | | |
| Diabetes | 13.3% | (10) | Y 11.60 ± 5.76 | −2.50 ± 2.46 | −0.90 ± 1.45 |
| | | | N 13.78 ± 8.34 | −2.76 ± 2.92 | −1.71 ± 2.51 |
| | | | p = 0.43 | p = 0.79 | p = 0.33 |
| Hypertension | 36.0% | (27) | Y 12.18 ± 7.02 | −3.56 ± 2.55 | −1.70 ± 2.88 |
| | | | N 14.02 ± 8.59 | −2.51 ± 2.87 | −1.34 ± 2.14 |
| | | | p = 0.35 | p = 0.12 | p = 0.54 |
| Depression | 45.3% | (34) | Y 13.41 ± 8.04 | −2.85 ± 2.59 | −1.56 ± 2.13 |
| | | | N 13.39 ± 8.08 | −2.85 ± 2.97 | −1.41 ± 2.63 |
| | | | p = 0.99 | p = 0.99 | p = 0.80 |
| PTSD | 30.7% | (23) | Y 14.30 ± 8.32 | −2.87 ± 3.18 | −1.48 ± 1.38 |
| | | | N 13.00 ± 7.91 | −2.85 ± 2.63 | −1.48 ± 2.75 |
| | | | p = 0.52 | p = 0.97 | p = 0.99 |
| Traumatic brain injury | 13.3% | (10) | Y 17.60 ± 11.66 | −2.60 ± 2.12 | −0.40 ± 1.84 |
| | | | N 12.64 ± 7.19 | −2.86 ± 2.90 | −1.63 ± 2.47 |
| | | | p = 0.069 | p = 0.79 | p = 0.14 |
| Sleep Apnea | 41.3% | (31) | Y 13.84 ± 8.31 | −3.06 ± 2.57 | −1.11 ± 1.89 |
| | | | N 13.00 ± 7.93 | −2.77 ± 2.96 | −1.74 ± 2.73 |
| | | | p = 0.66 | p = 0.65 | p = 0.21 |
| Hypercholesterolemia | 26.7% | (20) | Y 14.15 ± 7.15 | −3.85 ± 3.33 | −2.35 ± 2.60 |
| | | | N 13.10 ± 8.41 | −2.63 ± 2.50 | −1.15 ± 2.33 |
| | | | p = 0.62 | p = 0.10 | p = 0.06 |
| Autoimmune disease | 25.3% | (19) | Y 10.11 ± 7.27 | −3.37 ± 3.58 | −1.68 ± 1.77 |
| | | | N 14.52 ± 7.99 | −2.68 ± 2.48 | −1.41 ± 2.60 |
| | | | p = 0.04 | p = 0.36 | p = 0.67 |
| Sjögrens syndrome | 17.3% | (13) | Y 11.38 ± 7.47 | −3.38 ± 3.59 | −2.15 ± 1.81 |
| | | | N 14.30 ± 8.18 | −2.55 ± 2.52 | −1.36 ± 2.59 |
| | | | p = 0.24 | p = 0.33 | p = 0.30 |
| Eye drops | % (n) | | | | |
| Artificial tears | 77.3% | (58) | Y 13.41 ± 8.12 | −2.97 ± 2.91 | −1.53 ± 2.43 |
| | | | N 13.35 ± 8.83 | −2.47 ± 2.35 | −1.29 ± 2.39 |
| | | | p = 0.98 | p = 0.52 | p = 0.72 |
| Topical corticosteroids | 10.7% | (8) | Y 11.00 ± 7.95 | −3.13 ± 2.42 | −0.50 ± 1.77 |
| | | | N 13.83 ± 7.99 | −2.76 ± 2.82 | −1.56 ± 2.46 |
| | | | p = 0.35 | p = 0.73 | p = 0.24 |
| Cyclosporine 0.05% | 30.7% | (23) | Y 13.61 ± 8.25 | −3.13 ± 2.78 | −0.74 ± 1.82 |
| | | | N 13.49 ± 7.94 | −2.65 ± 2.77 | −1.76 ± 2.58 |
| | | | p = 0.95 | p = 0.49 | p = 0.09 |
| Lifitegrast | 8.0% | (6) | Y 15.00 ± 14.27 | −0.83 ± 3.87 | −1.50 ± 3.99 |
| | | | N 13.40 ± 7.35 | −2.97 ± 2.61 | −1.44 ± 2.26 |
| | | | p = 0.64 | p = 0.07 | p = 0.96 |
| Autologous Serum Tears | 9.3% | (7) | Y 9.00 ± 10.50 | −5.00 ± 2.71 | −2.71 ± 4.15 |
| | | | N 14.00 ± 7.62 | −2.57 ± 2.69 | −1.31 ± 2.15 |
| | | | p = 0.12 | p = 0.03 | p = 0.14 |

SD = standard deviation, PTSD = post-traumatic stress disorder; n = number; avg = average, PRT = phenol red thread; r = Pearson's correlation coefficient; F = female; M = male; W = white; B = black; H = Hispanic; NH = non-Hispanic; Y = yes; N = no; Δ = change; Significant p values in bold.

Change in tear volume: An increase in tear volume, as measured via PRT, was noted after one stimulation session (mean change 13.40±8.00 mm, p<0.0005). Some baseline measures significantly correlated with change in PRT. Specifically, patients with an autoimmune disease had less of an increase in volume compared to those without an autoimmune disease (10.11±7.27 vs. 14.52±7.99, p=0.04) (Table 2). Baseline tear volume was negatively correlated with change in volume; that is, individuals with lower baseline tear volume (i.e. ATD) had greater increases in volume with stimulation (r=−0.42) (Table 3). A multivariable forward step wise linear regression model that considered the contributions of demographics, medications, and DE measures found that baseline tear volume (standardized beta (β)=−0.501) and presence of an autoimmune disease (β=−0.355) remained significant predictors of change in tear volume with stimulation, R²=0.30.

TABLE 3
Dry eye parameters in the study population as they relate to change in tear
volume, dryness, and ocular pain after one nasal stimulation session, N = 75

| | Descriptive | | ΔTear volume | ΔDryness | ΔOcular pain |
|---|---|---|---|---|---|
| Eye symptoms, mean ± SD (range) | | | | | |
| Eye dryness immediately | 5.2 ± 2.6 | (0-10) | r = 0.02; | r = −0.30; | r = −0.25; |
| prior to stimulation (0-10) | | | p = 0.89 | p = 0.01 | p = 0.03 |
| Eye pain immediately prior | 4.04 ± 2.8 | (0-10) | r = −0.08; | r = −0.17; | r = −0.55; |
| to stimulation (0-10) | | | p = 0.50 | p = 0.15 | p = 0.0005 |
| DEQ5 (0-22) | 14.9 ± 3.8 | (4-22) | r = 0.07; | r = 0.15; | r = 0.003; |
| | | | p = 0.56 | p = 0.21 | p = 0.98 |
| OSDI (0-100) | 50.7 ± 22.1 | (0-100) | r = −0.08; | r = 0.009; | r = −0.07; |
| | | | p = 0.50 | p = 0.94 | p = 0.57 |
| Burning, avg 24 hours | 4.1 ± 3.1 | (0-10) | r = −0.05; | r = 0.18; | r = 0.04; |
| (0-10) | | | p = 0.70 | p = 0.11 | p = 0.71 |
| Pain evoked by wind, avg 24 | 4.8 ± 3.0 | (0-10) | r = −0.18; | r = 0.08; | r = −0.05; |
| hours (0-10) | | | p = 0.12 | p = 0.50 | p = 0.66 |
| Pain evoked by light, avg 24 | 5.1 ± 3.3 | (0-10) | r = −0.07; | r = 0.04; | r = −0.08; |
| hours (0-10) | | | p = 0.53 | p = 0.76 | p = 0.50 |
| Pain evoked by cold or hot, | 4.5 ± 3.3 | (0-10) | r = −0.02; | r = 0.11; | r = −0.01; |
| avg 24 hours (0-10) | | | p = 0.86 | p = 0.34 | p = 0.96 |
| Dry Eye Signs | | | | | |
| Pre-stimulation PRT | 16.01 ± 9.51 | (2-42) | r = −0.42; | r = 0.12; | r = 0.01; |
| | | | p < 0.0005 | p = 0.35 | p = 0.93 |
| InflammaDry (0-3) | 1.5 ± 1.0 | (0-3) | r = −0.16; | r = 0.15; | r = 0.08; |
| | | | p = 0.16 | p = 0.21 | p = 0.51 |
| Anterior blepharitis (0-3) | 0.6 ± 1.0 | (0-3) | r = −0.16; | r = −0.10; | r = −0.13; |
| | | | p = 0.18 | p = 0.40 | p = 0.25 |
| MG plugging (0-3) | 0.8 ± 0.8 | (0-3) | r = −0.11; | r = −0.05; | r = −0.23; |
| | | | p = 0.34 | p = 0.66 | p = 0.05 |
| Eyelid vascularity (0-3) | 1.2 ± 1.1 | (0-3) | r = −0.17; | r = −0.10; | r = −0.28; |
| | | | p = 0.15 | p = 0.39 | p = 0.01 |
| TBUT (seconds) | 5.7 ± 3.6 | (2-19) | r = 0.001; | r = 0.03; | r = −0.02; |
| | | | p = 0.99 | p = 0.82 | p = 0.88 |
| Cornea staining (0-15) | 3.7 ± 3.4 | (0-13) | r = −0.08; | r = 0.04; | r = −0.12; |
| | | | p = 0.51 | p = 0.73 | p = 0.31 |
| Temporal chalasis, % (n) | 65.3% | (49) | Y 13.36 ± 7.94 | −2.91 ± 2.94 | −1.96 ± 2.45 |
| | | | N 13.11 ± 8.45 | −2.77 ± 2.66 | −0.58 ± 2.18 |
| | | | p = 0.90 | p = 0.83 | p = 0.02 |
| Nasal chalasis, % (n) | 29.3% | (22) | Y 12.86 ± 9.45 | −2.50 ± 3.20 | −2.00 ± 2.96 |
| | | | N 13.62 ± 7.41 | −3.00 ± 2.62 | −1.26 ± 2.13 |
| | | | p = 0.71 | p = 0.48 | p = 0.23 |
| Persistent pain after | 53.3% | (40) | Y 13.40 ± 8.31 | −2.35 ± 2.92 | −1.10 ± 2.29 |
| anesthesia ≥ 1, % (n) | | | N 13.40 ± 7.83 | −3.43 ± 2.55 | −1.91 ± 2.49 |
| | | | p = 1.00 | p = 0.10 | p = 0.15 |
| Persistent pain after | 32.0% | (24) | 12.67 ± 8.32 | −0.13 ± 1.60 | −1.71 ± 2.74 |
| anesthesia ≥ 3, % (n) | | | 13.74 ± 7.91 | −2.12 ± 2.47 | −3.39 ± 2.66 |
| | | | p = 0.59 | p = 0.001 | p = 0.01 |
| Schirmer's test (mm | 11.2 ± 7.6 | (2-35) | r = −0.01; | r = −0.17; | r = −0.02; |
| wetting) | | | p = 0.93 | p = 0.14 | p = 0.85 |
| Meibum quality (0-4) | 2.1 ± 1.1 | (0-4) | r = −0.07; | r = 0.003; | r = −0.21; |
| | | | p = 0.56 | p = 0.98 | p = 0.07 |
| Dry Eye Categories | | | | | |
| Aqueous tear deficiency* | 28% | (21) | Y 16.33 ± 9.26 | −1.52 ± 2.04 | −3.23 ± 2.59 |
| | | | N 12.26 ± 7.24 | −1.46 ± 2.55 | −2.70 ± 2.87 |
| | | | p = 0.047 | p = 0.92 | p = 0.46 |
| Evaporative deficiency* | 33% | (25) | Y 10.44 ± 6.43 | −0.96 ± 1.51 | −2.28 ± 2.89 |
| | | | N 14.88 ± 8.36 | −1.74 ± 2.72 | −3.14 ± 2.72 |
| | | | p = 0.02 | p = 0.18 | p = 0.21 |

SD = standard deviation,
n = number;
avg = average,
PRT = phenol red thread;
r = Pearson's correlation coefficient;
DEQ5 = Dry Eye Questionnaire 5;
OSDI = Ocular Surface Disease Index;
MMP-9 = matrix metalloproteinase 9;
MG = Meibomian gland;
TBUT = Tear break up time;
Δ = change
*Aqueous tear deficiency (ATD) was defined as a PRT value of <10 mm in either eye prior to stimulation and evaporative deficiency as a PRT value of ≥10 mm in both eyes and TBUT <5 seconds in either eye.
Significant p values in bold.

Change in ocular dryness and pain: Dryness and ocular pain intensities were both reduced after neurostimulation (mean change −2.85±2.79 for dryness and −1.48±2.41 for pain, p<0.0005 for both). Sixty-five percent of individuals had a ≥30% reduction in dryness intensity after neurostimulation and 59% had ≥50% reduction. Likewise, 43% of individuals had ≥30% reduction in ocular pain intensity and 37% had a ≥50% reduction. Nineteen (25%) individuals reported no change in dryness after stimulation and 2 (2.6%) reported an intensification of dryness. Similarly, 24 (32%) reported no change in pain after stimulation and 5 (6.7%) reported an intensification of pain.

Individuals with lower baseline dryness scores had a greater improvement in dryness sensations with neurostimulation (r=−0.30). Younger age (r=−0.30), lower baseline dryness (r=−0.25) and eye pain scores (r=−0.30), and eyelid vascularity (r=−0.28) correlated with greater improvements in ocular pain. Individuals with moderate persistent pain after anesthesia (≥3) had less of an improvement in sensations of dryness and pain with neurostimulation compared to those with no or mild pain.

A multivariable forward step wise linear regression model considering the contributions of demographics and DE measures on change in subjective metrics found that baseline ocular dryness score (β=−0.55), moderate persistent pain after anesthesia (β=0.39), and Inflammadry score (β=0.21) predicted change in dryness sensation with neurostimulation, R²=0.64. Baseline ocular pain score (β=−0.79), moderate persistent pain after anesthesia (β=0.62), and age (β=−0.16) predicted change in ocular pain score, R²=0.72. Interestingly, change in self-reported dryness and ocular pain were not related to change in tear volume (dryness r=−0.13, p=0.25; ocular pain r=0.07, p=0.56).

Subjective assessment of device: The majority of individuals (65%) rated the device as “easy” or “very easy” to use while 16% rated it as “difficult”. The majority of individuals (81%) indicated that their eyes felt “slightly” or “much” better after treatment while 4% felt worse. Finally, 56% reported that they would use the device “frequently” at home. No complications related to the neurostimulation session were noted.

### Discussion

Described herein are the effects of one TrueTear® session on painful DE symptoms. It was found that an overall reduction in sensations of dryness and ocular pain in the entire group was not correlated with change in tear volume. Individuals with lower baseline symptom intensities had greater reduction in symptoms with stimulation. Conversely, those with moderate pain after placement of topical anesthesia had less of an improvement in symptoms with neurostimulation compared to those with no or mild pain. These findings suggest that mechanisms beyond tear volume contribute to ocular sensations and that high pain levels may be less modifiable by neurostimulation of the anterior ethmoidal nerve.

It was also tested whether change in tear volume after a neurostimulation session could be used as a surrogate measure of nasolacrimal arc reactivity. More specifically, it was assessed whether individuals with symptoms consistent with neuropathic pain (e.g. burning, evoked pain to wind and light, and persistent pain after anesthesia[23]) would demonstrate a greater increase in tear volume, reflective of a more sensitive nasolacrimal arc. The results, however, do not support this notion as the above noted factors did not associate with change in tear volume. On the other hand, the presence of a systemic autoimmune disease was associated with less of a volume increase after neurostimulation. This is not surprising as this subset of patients are more likely to have lacrimal gland abnormalities.

With respect to tear stimulation, the results described herein are similar to prior TrueTear® studies. The results show a mean increase of 13.4 in tear volume (via PRT) in the study population described herein. In other studies, tear production via Schirmer increased by a mean of 16.1 mm[8] and 13 mm[15] after one intranasal neurostimulation session and was noted across a range of severity categories.[15] Interestingly, in a previous study, the increase in tear production was most robust during the first stimulation session and decreased at subsequent visits (day 7, 14, 30, and 90).[14] Overall, other DE signs, including TBUT and conjunctival staining, were similar between the active versus sham neurostimulation groups at the time points tested, the exception being corneal staining at 90 days.[14]

Given the disconnect between reduced ocular pain and increased tear volume, it was tested whether TrueTear® may act in a manner similar to transcutaneous electrical simulation (TENS). Electrical stimulation is an accepted treatment approach in chronic pain that has been applied to oral and facial pain conditions.[24-28] In a pilot study of 14 individuals, non-invasive peripheral stimulation of trigeminal nerve V1 branches with a RS4i (RS Medical, Vancouver, Wash.) improved ocular pain and light sensitivity in 14 individuals after one 30 minute session. [29] The magnitude of effect was higher (2.62±0.68 reduction in pain intensity in the right eye) compared to the 1.48±2.41 reduction noted with TrueTear®. While the electrical stimuli are not identical between the two devices, including a shorter duration (seconds vs 30 min), lower amplitude (1.5 mA vs ˜50 mA), and lower frequency (30-60 Hz vs 100 Hz) in TrueTear® compared to RS4i[29], there may be shared mechanisms of pain reduction. One potential explanation is that stimulating sensory nerves reduces pain by blocking nociceptive signaling, a concept known as Gate-Control Theory of Pain. [30] As applied to TrueTear®, stimulation of large AP fibers in the anterior ethmoidal nerve may presynaptically inhibit corneal small C fibers from exciting 2^{nd} order neurons in the spinal trigeminal nucleus, thus reducing the sensation of pain in the somatosensory cortex (FIG. 1). Other potential shared mechanisms of pain reduction in TENS include increases in endogenous opioids, activation of inhibitory descending signals from the periaqueductal gray (PAG), and decreases in excitatory neurotransmitters such as glutamate and substance P.[31] The smaller degree of pain reduction noted in those with high baseline ocular pain intensities point to the possibility that less modifiable, central mechanisms play a role in mediating pain in these individuals.

FIG. 1 shows a hypothesized pathway for TrueTear® pain modulation and simultaneous stimulation of nasolacrimal pathway. Corneal nociceptive signals travel through afferent C fibers in the V1 branch of the trigeminal nerve and synapse with 2nd order neurons in the spinal trigeminal nucleus (STN) of the brainstem. The 2nd order neuron synapse in the thalamus with 3^{rd} order thalamo-cortical neurons that mediate pain sensation in the somatosensory cortex (red line). TrueTear® stimulates the nasolacrimal pathway beginning with the anterior ethmoidal nerve branch of V1 (green line) which synapses on an excitatory interneuron in the STN. We hypothesize that stimulation of large AP fibers in the anterior ethmoidal nerve may presynaptically inhibit corneal small C fibers from activating 2nd order neurons in the spinal trigeminal nucleus, thus reducing the sensation of pain in the somatosensory cortex (red line). At the same time, the interneuron in the STN activates the efferent pre-ganglionic parasympathetic neuron in the superior salivatory nucleus (SSN), which travels through the facial nerve (CN VII) and the geniculate ganglion (GN) to synapse with the post-ganglionic parasympathetic neuron cell body in the pterygopalantine ganglion (PPG). The post-ganglionic parasympathetic neurons continue through the zygomatic branch of V2 and then the lacrimal nerve of V1 to induce a tearing response (blue line).

The findings described herein included a patient population recruited from a DE clinic with a predominant male majority. As such, these findings may not be generalizable to DE populations with a predominantly female population. However, a strength of the study described herein was including patients with a variety of DE symptoms and signs. The ocular surface examination also did not include assessment of tear osmolarity or corneal nerve anatomy and these factors may have correlated with the outcome measures. Furthermore, information on systemic disease severity in individuals with auto-immune conditions was not collected. Finally, one intranasal neurostimulation session was performed in the clinical setting and it is not known whether the findings described herein would be reproducible after multiple sessions. Despite these limitations, described herein is information on responses to one session of TrueTear® with regards to subjective and objective measures of DE. It was found that overall, tear volume increased and reported ocular dryness and pain intensity decreased after one session. However, these effects were independent of one another, suggesting that improving tear production alone may not improve DE symptoms in each patient. Overall, the results reported in this study can help identify individuals more likely to have a positive response to intranasal stimulation therapy.

### REFERENCES

1. Stapleton F, Alves M, Bunya V Y, et al. TFOS DEWS II Epidemiology Report. Ocul Surf 2017; 15:334-65.
2. Craig J P, Nichols K K, Akpek E K, et al. TFOS DEWS II Definition and Classification Report. Ocul Surf 2017; 15:276-83.
3. Jones L, Downie L E, Korb D, et al. TFOS DEWS II Management and Therapy Report. Ocul Surf 2017; 15:575-628.
4. Galor A, Moein H R, Lee C, et al. Neuropathic pain and dry eye. Ocul Surf 2017.
5. Aggarwal S, Kheirkhah A, Cavalcanti B M, et al. Autologous Serum Tears for Treatment of Photoallodynia in Patients with Corneal Neuropathy: Efficacy and Evaluation with In Vivo Confocal Microscopy. Ocul Surf 2015; 13:250-62.
6. Diel R J, Kroeger Z A, Levitt R C, et al. Botulinum Toxin A for the Treatment of Photophobia and Dry Eye. Ophthalmology 2018; 125:139-40.
7. Diel R J, Hwang J, Kroeger Z A, et al. Photophobia and sensations of dryness in patients with migraine occur independent of baseline tear volume and improve following botulinum toxin A injections. Br J Ophthalmol 2019; 103:1024-29.
8. Sheppard J D, Torkildsen G L, Geffin J A, et al. Characterization of tear production in subjects with dry eye disease during intranasal tear neurostimulation: Results from two pivotal clinical trials. Ocul Surf 2019; 17:142-50.
9. Zilstorff-Pedersen K. Quantitative Measurements of the Nasolacrimal Reflex. Arch Otolaryngol 1965; 81:457-62.
10. Belmonte C, Nichols J J, Cox S M, et al. TFOS DEWS II pain and sensation report. Ocul Surf 2017; 15:404-37.
11. Ding C, Walcott B, Keyser K T. Sympathetic neural control of the mouse lacrimal gland. Invest Ophthalmol Vis Sci 2003; 44:1513-20.
12. Brinton M, Kossler A L, Patel Z M, et al. Enhanced Tearing by Electrical Stimulation of the Anterior Ethmoid Nerve. Invest Ophthalmol Vis Sci 2017; 58:2341-48.
13. Gumus K, Schuetzle K L, Pflugfelder S C. Randomized Controlled Crossover Trial Comparing the Impact of Sham or Intranasal Tear Neurostimulation on Conjunctival Goblet Cell Degranulation. Am J Ophthalmol 2017; 177:159-68.
14. Cohn G S, Corbett D, Tenen A, et al. Randomized, Controlled, Double-Masked, Multicenter, Pilot Study Evaluating Safety and Efficacy of Intranasal Neurostimulation for Dry Eye Disease. Invest Ophthalmol Vis Sci 2019; 60:147-53.
15. Friedman N J, Butron K, Robledo N, Loudin J, Baba S N, Chayet A. A nonrandomized, open-label study to evaluate the effect of nasal stimulation on tear production in subjects with dry eye disease. Clin Ophthalmol 2016; 10:795-804.
16. Galor A, Moein H R, Lee C, et al. Neuropathic pain and dry eye. Ocul Surf 2018; 16:31-44.
17. Schiffman R M, Christianson M D, Jacobsen G, Hirsch J D, Reis B L. Reliability and validity of the Ocular Surface Disease Index. Arch Ophthalmol 2000; 118:615-21.
18. Chalmers R L, Begley C G, Caffery B. Validation of the 5-Item Dry Eye Questionnaire (DEQ-5): Discrimination across self-assessed severity and aqueous tear deficient dry eye diagnoses. Cont Lens Anterior Eye 2010; 33:55-60.
19. Vashisht S, Singh S. Evaluation of Phenol Red Thread test versus Schirmer test in dry eyes: A comparative study. Int J Appl Basic Med Res 2011; 1:40-2.
20. Galor A, Feuer W, Lee D J, Florez H, Venincasa V D, Perez V L. Ocular surface parameters in older male veterans. Invest Ophthalmol Vis Sci 2013; 54:1426-33.
21. Wolffsohn J S, Arita R, Chalmers R, et al. TFOS DEWS II Diagnostic Methodology report. Ocul Surf 2017; 15:539-74.
22. Crane A M, Feuer W, Felix E R, et al. Evidence of central sensitisation in those with dry eye symptoms and neuropathic-like ocular pain complaints: incomplete response to topical anaesthesia and generalised heightened sensitivity to evoked pain. Br J Ophthalmol 2017; 101:1238-43.
23. Farhangi M, Feuer W, Galor A, et al. Modification of the Neuropathic Pain Symptom Inventory for use in eye pain (NPSI-Eye). Pain 2019; 160:1541-50.
24. Kasat V, Gupta A, Ladda R, Kathariya M, Saluj a H, Farooqui A-A. Transcutaneous electric nerve stimulation (TENS) in dentistry-A review. Journal of clinical and experimental dentistry 2014; 6:e562.
25. Johnson M D, Burchiel K J. Peripheral stimulation for treatment of trigeminal postherpetic neuralgia and trigeminal posttraumatic neuropathic pain: a pilot study. Neurosurgery 2004; 55:135-42.
26. Holsheimer J. Electrical stimulation of the trigeminal tract in chronic, intractable facial neuralgia. Archives of physiology and biochemistry 2001; 109:304-08.
27. Eriksson M B, Sjolund B H, Sundbarg G. Pain relief from peripheral conditioning stimulation in patients with chronic facial pain. Journal of neurosurgery 1984; 61:149-55.
28. Hansson P, Ekblom A. Transcutaneous electrical nerve stimulation (TENS) as compared to placebo TENS for the relief of acute oro-facial pain. Pain 1983; 15:157-65.
29. Sivanesan E, Levitt R C, Sarantopoulos C D, Patin D, Galor A. Noninvasive Electrical Stimulation for the Treatment of Chronic Ocular Pain and Photophobia. Neuromodulation 2018; 21:727-34.
30. Mendell L M. Constructing and deconstructing the gate theory of pain. Pain 2014; 155:210-6.
31. Vance C G, Dailey D L, Rakel B A, Sluka K A. Using TENS for pain control: the state of the evidence. Pain Manag 2014; 4:197-209.

1. A method comprising: stimulating nasal tissue of a subject, wherein stimulating nasal tissue of the subject:
a. reduces ocular pain in the subject;
b. treats ocular pain in the subject;
c. reduces ocular burning and/or ocular stinging in the subject; and/or
d. reduces or ameliorates one or more signs, symptoms, causes or effects of ocular pain in the subject.

2. The method of claim 1, wherein stimulating nasal tissue of the subject reduces ocular pain in the subject, and wherein reduction of the ocular pain in the subject is not associated with a change in tear volume compared to the tear volume in the subject before stimulation.

3. The method of claim 1, wherein stimulating the nasal tissue of the subject reduces ocular burning and/or ocular stinging in the subject.

4. The method of claim 1, wherein stimulating the nasal tissue of the subject reduces or ameliorates one or more signs, symptoms, causes or effects of ocular pain in the subject.

5. The method of claim 4, wherein the one or more signs, symptoms causes, or effects of ocular pain are burning, stinging, aching, scratching, itching, redness, inflammation, discharge, headache, light sensitivity, visual disturbances, tearing, or a combination thereof.

6. The method of claim 4, wherein the one or more signs, symptoms, causes or effects are selected from the group consisting of impaired vision, burning sensation, redness, irritation, inflammation, engorged vasculature, anterior lid margin vascularization, zone A posterior lid margin vascularization, eyelid disorders, swelling, vital staining, Schirmer's score, or Meibomian gland obstruction.

7. The method claim 1, wherein stimulating the nasal tissue of the subject treats for treating ocular pain in the subject.

8. The method of claim 7, wherein the ocular pain in the subject is reduced and is not associated with a change in tear volume compared to the tear volume in the subject before stimulation.

9. The method of claim 1, further comprising identifying the subject in need of treatment.

10. The method of claim 9, wherein the identifying step comprises detecting a sign or symptom selected from the group consisting of scratching, stinging, itching, burning, redness, inflammation, discharge, headache, light sensitivity, aching, visual disturbances, or tearing.

11. The method of claim 9, wherein the subject does not have low tear volume.

12. The method of claim 11, wherein the subject is a human having a Schirmer's score of greater than 10 mm before stimulation of the nasal tissue of the subject.

13. The method of claim 12, wherein the subject does not have dry eye disease.

14. The method of claim 1, wherein the subject has a condition that comprises one or more of or has been diagnosed with one or more of: orofacial pain, macular degeneration, glaucoma, cataracts, optic neuritis, corneal disorders, corneal abrasions, iritis, uveitis, sinusitis, cluster headache, migraine, corneal ulcer, multiple sclerosis, blepharitis, meibomitis gland dysfunction, an autoimmune disease and diabetic retinopathy.

15. The method of claim 14, wherein the autoimmune disease is Sjögren's, multiple sclerosis, rheumatoid arthritis, or psoriatic arthritis.

16. The method of claim 1, further comprising administering topical cyclosporine, topical interleukin-1, or a combination thereof.

17. The method of claim 17, wherein cyclosporine or interleukin-1 are administered at a concentration of 2.5% to 5%.

18. The method of claim 1, wherein stimulating nasal tissue of the subject comprises inserting a first nasal insertion prong of a stimulator probe having a first electrode into a first nostril of a nose of the subject and inserting a second nasal insertion prong of the stimulator probe having a second electrode into a second nostril of the nose, such that the first and second electrodes are positioned adjacent to a septum of the subject, wherein the stimulator probe is connected to a stimulator body, and wherein the stimulator body comprises a control subsystem to control an electrical stimulus to be delivered to the subject via the stimulator probe.

19. The method of claim 18, wherein the method further comprises: delivering the stimulus to activate a nerve, wherein the stimulus has a maximum amplitude between 10 μA and 100 mA and is delivered in a bipolar configuration between the first and second electrodes.

20. The method of claim 19, wherein the stimulus comprises a waveform having a frequency between 20 Hz and 80 Hz.

21-23. (canceled)
